(19) United States
(12) Patent Application Publication
(10) Pub. No.: US 2024/0382180 A1
(43) Pub. Date: Nov. 21, 2024

(54) ALIGNMENT FOR MULTIPLE SERIES OF INTRAVASCULAR IMAGES

(71) Applicant: Boston Scientific Scimed Inc., Maple Grove, MN (US)

(72) Inventors: Alexander Shang, Hopkins, MN (US); Wenguang Li, Los Gatos, CA (US); Kevin Bloms, Minneapolis, MN (US); Yan Li, Plymouth, MN (US)

(73) Assignee: Boston Scientific Scimed Inc., Maple Grove, MN (US)

(21) Appl. No.: 18/667,989

(22) Filed: May 17, 2024

Related U.S. Application Data
(60) Provisional application No. 63/648,483, filed on May 16, 2024.
(60) Provisional application No. 63/502,859, filed on May 17, 2023.

(51) Int. Cl. A61B 8/08; A61B 8/00; A61B 8/12

(52) U.S. Cl. CPC A61B 8/5207; A61B 8/0891; A61B 8/12; A61B 8/463; A61B 8/5246

(57) ABSTRACT

The present disclosure provides to process intravascular ultrasound (IVUS) images from different runs through a vessel to generate a mapping between frames of each IVUS run and to generate a graphical user interface (GUI) to graphically present the IVUS runs in relationship to each other. In some examples, a vessel fiducial is identified in a frame of each IVUS run and one or both runs are offset in time, distance, and/or angle to align the frames with the identified vessel fiducial. Further, the disclosure provides to angularly align intravascular images to a viewing perspective of an external image of the vessel.

### CROSS-REFERENCE TO RELATED APPLICATIONS

This application claims the benefit of U.S. Provisional Patent Application Ser. No. 63/648,483 filed on May 16, 2024 and U.S. Provisional Patent Application Ser. No. 63/502,859 filed on May 17, 2023, the disclosures of which are incorporated herein by reference.

### TECHNICAL FIELD

The present disclosure generally relates to intravascular ultrasound (IVUS) imaging system. Particularly, but not exclusively, the present disclosure relates to correlating frames in a first series of IVUS images with frames in a second series of IVUS images.

### BACKGROUND

Ultrasound devices insertable into patients have proven diagnostic capabilities for a variety of diseases and disorders. For example, intravascular ultrasound (IVUS) imaging systems have been used as an imaging modality for diagnosing blocked blood vessels and providing information to aid medical practitioners in selecting and placing stents and other devices to restore or increase blood flow.

IVUS imaging systems include a control module (with a pulse generator, an image acquisition and processing components, and a monitor), a catheter, and a transducer disposed in the catheter. The transducer-containing catheter is positioned in a lumen or cavity within, or in proximity to, a region to be imaged, such as a blood vessel wall or patient tissue in proximity to a blood vessel wall. The pulse generator in the control module generates electrical pulses that are delivered to the transducer and transformed to acoustic pulses that are transmitted through patient tissue. The patient tissue (or other structure) reflects the acoustic pulses and reflected pulses are absorbed by the transducer and transformed to electric pulses. The transformed electric pulses are delivered to the image acquisition and processing components and converted into images displayable on the monitor.

Often, physicians will capture a series of IVUS images at different stages of treatment. However, conventional tools and systems to not allow the physician to compare these different series of IVUS images besides providing a select set of measurements taken from the images. Thus, there is a need to correlate IVUS images of the same vessel taken at different times and provide a graphical interface to display these images in relation to each other.

Machine learning (ML) is the study of computer algorithms that improve through experience. Typically, ML algorithms build a model based on sample data, referred to as training data. The model can be used to infer (e.g., make predictions or decisions without explicitly being programmed to do so. As will be appreciated, the quality of the inference a model makes is dependent upon the training data. Thus, there is a need to provide a larger and more complete corpus of knowledge with which these ML models are trained.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In general, the present disclosure is provided to process raw IVUS images, automatically detected lumen and vessel borders, and identify regions of interest, or more particularly, starting and ending points between which include frames of interest in a series of IVUS images.

In some embodiments, the disclosure can be implemented as a method for a computing device. The method can comprise receiving, by a processor, a first series of intravascular ultrasound (IVUS) images of a vessel of a patient, the first series of IVUS images comprising a first plurality of frames; receiving, by the processor, a second series of intravascular ultrasound (IVUS) images of the vessel of the patient, the second series of IVUS images comprising a second plurality of frames; determining, by the processor, an offset for the first plurality of frames based at least in part on the second plurality of frames; applying, by the processor, the offset to the first plurality of frames to generate an offset series of IVUS images; and generating, by the processor, a graphical user interface (GUI), the GUI comprising indications of the offset series of IVUS images and the second series of IVUS images.

In further embodiments of the method, determining the offset for the first plurality of frames comprises: identifying a frame of the first plurality of frames comprising a vessel fiducial; identifying a frame of the second plurality of frames comprising the vessel fiducial; and determining the offset for the first plurality of frames that when applied aligns the frame of the first plurality of frames comprising the vessel fiducial with the frame of the second plurality of frames comprising the vessel fiducial.

In further embodiments of the method, the offset comprises a first offset and a second offset and wherein determining the offset for the first plurality of frames based comprises: identifying a first frame of the first plurality of frames comprising a first vessel fiducial; identifying a second frame of the second plurality of frames comprising the first vessel fiducial; determining the first offset for the first plurality of frames that when applied to a first segment of the first plurality of frames aligns the first frame of the first plurality of frames with the first frame of the second plurality of frames; identifying a second frame of the first plurality of frames comprising a second vessel fiducial; identifying a second frame of the second plurality of frames comprising the second vessel fiducial; and determining the second offset for the first plurality of frames that when applied to a second segment of the first plurality of frames different than the first segment, aligns the second frame of the first plurality of frames with the second frame of the second plurality of frames, wherein the second offset is different from the first offset.

In further embodiments of the method, the first offset comprises an offset distance and the second offset comprises an offset angle or wherein the first offset comprises an offset distance or an offset angle and the second offset comprises an offset distance and an offset angle.

In further embodiments of the method, identifying the frame of the first plurality of frames comprising the vessel fiducial and wherein identifying the frame of the second plurality of frames comprising the vessel fiducial comprises: executing a machine learning (ML) model to infer the frame of the first plurality of frames comprising the vessel fiducial; and executing the ML model to infer the frame of the second plurality of frames comprising the vessel fiducial.

In further embodiments of the method, the vessel fiducial is one of a lumen geometry, a vessel geometry, a side branch location, a calcium morphology, a plaque distribution, or a guide catheter position.

In further embodiments of the method, determining the offset for the first plurality of frames comprises: calculating a correlation score for each frame of the first plurality of frames based on a frame-by-frame correlation with the second plurality of frames; identifying a frame of the first plurality of frames having the highest correlation score and a frame of the second plurality of frames associated with the highest correlation score; and determining the offset for the first plurality of frames that when applied aligns the frame of the first plurality of frames with the highest correlation score with the frame of the second plurality of frames associated with the highest correlation score.

In further embodiments of the method, the offset is an offset distance and wherein the method further comprises: calculating a correlation score for each frame of the first plurality of frames based on an angular offset frame-by-frame correlation with the second plurality of frames; identifying a frame of the first plurality of frames having the highest correlation score and a frame or a rotated version of the frame of the second plurality of frames associated with the highest correlation score; and determining an offset angle for the first plurality of frames based on the frame or the rotated version of the frame of the second plurality of frames associated with the highest correlation score that when applied aligns the frame of the first plurality of frames with the highest correlation score with the frame of the second plurality of frames associated with the highest correlation score, wherein the offset series of IVUS images is generated by applying the offset distance and the offset angle to the first plurality of frames.

In further embodiments of the method, determining the offset for the first plurality of frames comprises: calculating a correlation score for each frame of the first plurality of frames based on an angular offset frame-by-frame correlation with the second plurality of frames; identifying a frame of the first plurality of frames having the highest correlation score and a frame or a rotated version of the frame of the second plurality of frames associated with the highest correlation score; and determining the offset for the first plurality of frames based on the frame or the rotated version of the frame of the second plurality of frames associated with the highest correlation score that when applied aligns the frame of the first plurality of frames with the highest correlation score with the frame of the second plurality of frames associated with the highest correlation score.

In further embodiments of the method, the offset for the first plurality of frames is a distance offset, an angle offset, or a distance and an angle offset.

In further embodiments, the method can comprise receiving the second series of IVUS images from an intravascular imaging device; and receiving the first series of IVUS images from a memory storage device.

In further embodiments of the method, the first series of IVUS images are captured during a pre-percutaneous coronary intervention (PCI) procedure.

In further embodiments of the method, the second series of IVUS images are captured during a peri-PCI or post-PCI procedure.

In further embodiments of the method, the GUI comprises a longitudinal view of the first series of IVUS images and the second series of IVUS images and wherein the longitudinal views are set against a common scale.

With some embodiments, the disclosure can be implemented as an apparatus for an intravascular imaging system. The apparatus can comprise a processor; and a memory device coupled to the processor, the memory device comprising instructions executable by the processor, which instructions when executed by the processor cause the intravascular imaging system to implement any of the methods outlined herein.

With some embodiments, the disclosure can be implemented as at least one machine readable storage device. The at least one machine readable storage device can comprise a plurality of instructions that in response to being executed by a processor of an intravascular ultrasound (IVUS) imaging system cause the processor to implement any of the methods outlined herein.

With some embodiments, the disclosure can be implemented as an apparatus for an intravascular imaging system. The apparatus can comprise a 16. An apparatus for an intravascular imaging system, comprising a display; a processor coupled to the display; and a memory device coupled to the processor, the memory device comprising instructions executable by the processor, which instructions when executed by the processor cause the intravascular imaging system to receive a first series of intravascular ultrasound (IVUS) images of a vessel of a patient, the first series of IVUS images comprising a first plurality of frames; receive a second series of intravascular ultrasound (IVUS) images of the vessel of the patient, the second series of IVUS images comprising a second plurality of frames; determine an offset for the first plurality of frames based at least in part on the second plurality of frames; apply the offset to the first plurality of frames to generate an offset series of IVUS images; generate a graphical user interface (GUI), the GUI comprising indications of the offset series of IVUS images and the second series of IVUS images; and display the GUI on the display.

In further embodiments of the apparatus, the instructions further cause the intravascular imaging system to identify a frame of the first plurality of frames comprising a vessel fiducial; identify a frame of the second plurality of frames comprising the vessel fiducial; and determine the offset for the first plurality of frames that when applied aligns the frame of the first plurality of frames comprising the vessel fiducial with the frame of the second plurality of frames comprising the vessel fiducial.

In further embodiments of the apparatus, the offset comprises a first offset and a second offset and wherein the instructions further cause the intravascular imaging system to: identify a first frame of the first plurality of frames comprising a first vessel fiducial; identify a second frame of the second plurality of frames comprising the first vessel fiducial; determine the first offset for the first plurality of frames that when applied to a first segment of the first plurality of frames aligns the first frame of the first plurality of frames with the first frame of the second plurality of frames; identify a second frame of the first plurality of frames comprising a second vessel fiducial; identify a second frame of the second plurality of frames comprising the second vessel fiducial; and determine the second offset for the first plurality of frames that when applied to a second segment of the first plurality of frames different than the first segment, aligns the second frame of the first plurality of frames with the second frame of the second plurality of frames, wherein the second offset is different from the first offset.

In further embodiments of the apparatus, the first offset comprises an offset distance and the second offset comprises an offset angle or wherein the first offset comprises an offset distance or an offset angle and the second offset comprises an offset distance and an offset angle.

In further embodiments of the apparatus, the instructions further cause the intravascular imaging system to: execute a machine learning (ML) model to infer the frame of the first plurality of frames comprising the vessel fiducial; and execute the ML model to infer the frame of the second plurality of frames comprising the vessel fiducial.

In further embodiments of the apparatus, the vessel fiducial is one of a lumen geometry, a vessel geometry, a side branch location, a calcium morphology, a plaque distribution, or a guide catheter position.

With some embodiments, the disclosure can be implemented as at least one machine readable storage device. The at least one machine readable storage device can comprise a plurality of instructions that in response to being executed by a processor of an intravascular ultrasound (IVUS) imaging system cause the processor to receive a first series of intravascular ultrasound (IVUS) images of a vessel of a patient, the first series of IVUS images comprising a first plurality of frames; receive a second series of intravascular ultrasound (IVUS) images of the vessel of the patient, the second series of IVUS images comprising a second plurality of frames; determine an offset for the first plurality of frames based at least in part on the second plurality of frames; apply the offset to the first plurality of frames to generate an offset series of IVUS images; generate a graphical user interface (GUI), the GUI comprising indications of the offset series of IVUS images and the second series of IVUS images; and send the GUI to a display coupled to the IVUS imaging system.

In further embodiments of the at least one machine readable storage device, execution of the instructions further causes the IVUS imaging system to calculate a correlation score for each frame of the first plurality of frames based on a frame-by-frame correlation with the second plurality of frames; identify a frame of the first plurality of frames having the highest correlation score and a frame of the second plurality of frames associated with the highest correlation score; and determine the offset for the first plurality of frames that when applied aligns the frame of the first plurality of frames with the highest correlation score with the frame of the second plurality of frames associated with the highest correlation score.

In further embodiments of the at least one machine readable storage device, the offset is an offset distance and execution of the instructions further causes the IVUS imaging system to calculate a correlation score for each frame of the first plurality of frames based on an angular offset frame-by-frame correlation with the second plurality of frames; identify a frame of the first plurality of frames having the highest correlation score and a frame or a rotated version of the frame of the second plurality of frames associated with the highest correlation score; and determine an offset angle for the first plurality of frames based on the frame or the rotated version of the frame of the second plurality of frames associated with the highest correlation score that when applied aligns the frame of the first plurality of frames with the highest correlation score with the frame of the second plurality of frames associated with the highest correlation score, wherein the offset series of IVUS images is generated by applying the offset distance and the offset angle to the first plurality of frames.

In further embodiments of the at least one machine readable storage device, execution of the instructions further causes the IVUS imaging system to calculate a correlation score for each frame of the first plurality of frames based on an angular offset frame-by-frame correlation with the second plurality of frames; identify a frame of the first plurality of frames having the highest correlation score and a frame or a rotated version of the frame of the second plurality of frames associated with the highest correlation score; and determine the offset for the first plurality of frames based on the frame or the rotated version of the frame of the second plurality of frames associated with the highest correlation score that when applied aligns the frame of the first plurality of frames with the highest correlation score with the frame of the second plurality of frames associated with the highest correlation score.

In further embodiments of the at least one machine readable storage device, the offset for the first plurality of frames is a distance offset, an angle offset, or a distance and an angle offset.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates an IVUS imaging system in accordance with embodiments of the disclosure.

FIG. 2 illustrates an example angiogram image of a vessel.

FIG. 3A and FIG. 3B illustrate IVUS images of the vessel.

FIG. 4 illustrates IVUS images correlation and visualization system, in accordance with at least one embodiment of the present disclosure.

FIG. 5A illustrates an example frame-by-frame correlation between a frame in a set of IVUs images and frames in another set of IVUS images, in accordance with at least one embodiment of the present disclosure.

FIG. 5B illustrates a plot of the correlation score that can be generated according to the example frame-by-frame correlation in FIG. 5A.

FIG. 6A illustrates another example frame-by-frame correlation between a frame in a set of IVUs images and a frame and angularly offset versions of the frame from another set of IVUS images, in accordance with at least one embodiment of the present disclosure.

FIG. 6B illustrates a plot of the correlation score that can be generated according to the example frame-by-frame correlation in FIG. 6A.

FIG. 7 illustrates another IVUS images correlation and visualization system, in accordance with at least one embodiment of the present disclosure.

FIGS. 8A and 8B illustrate several series of IVUS images of a vessel aligned based on at least one embodiment of the present disclosure.

FIG. 9A and FIG. 9B illustrate an example segment-by-segment alignment and offset identification, in accordance with at least one embodiment of the present disclosure.

FIG. 10A and FIG. 10B illustrate another example segment-by-segment alignment and offset identification, in accordance with at least one embodiment of the present disclosure.

FIG. 11 illustrates a graphical user interface (GUI) in accordance with at least one embodiment of the present disclosure.

FIG. 12 illustrates a logic flow to determine a mapping between different IVUS runs of a vessel in accordance with at least one embodiment of the present disclosure.

FIG. 13 illustrates time warping along a longitudinal offset of two series of IVUS images based on extracted and vectorized features of the series of IVUS images according to at least one embodiment of the present disclosure.

FIG. 14 illustrates an exemplary machine learning ML system suitable for use with exemplary embodiments of the present disclosure.

FIG. 15 illustrates another IVUS images correlation and visualization system, in accordance with at least one embodiment of the present disclosure.

FIG. 16A illustrates an example extravascular image and identified fiducials.

FIG. 16B, FIG. 16C, and FIG. 16D illustrate examples of frames of an IVUS run rotated to be aligned with the angle of the fiducials as viewed in the external image of FIG. 16A.

FIG. 17 illustrates a GUI in accordance with at least one embodiment of the present disclosure.

FIG. 18 illustrates a computer-readable storage medium.

FIG. 19 illustrates a diagrammatic representation of a machine.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and operation, together with further objects and advantages will be better understood from the following description when considered in connection with the accompanying figures. It is to be expressly understood, however, that each of the figures is provided for the purpose of illustration and description and is not intended as a definition of the limits of the present disclosure.

As noted, the present disclosure relates to IVUS images and lumens (e.g., vessels) of patients and to processing an IVUS recording, or said differently, processing a series of IVUS images. As such, an example IVUS imaging system, patient vessel, and series of IVUS images is described.

Suitable IVUS imaging systems include, but are not limited to, one or more transducers disposed on a distal end of a catheter configured and arranged for percutaneous insertion into a patient.

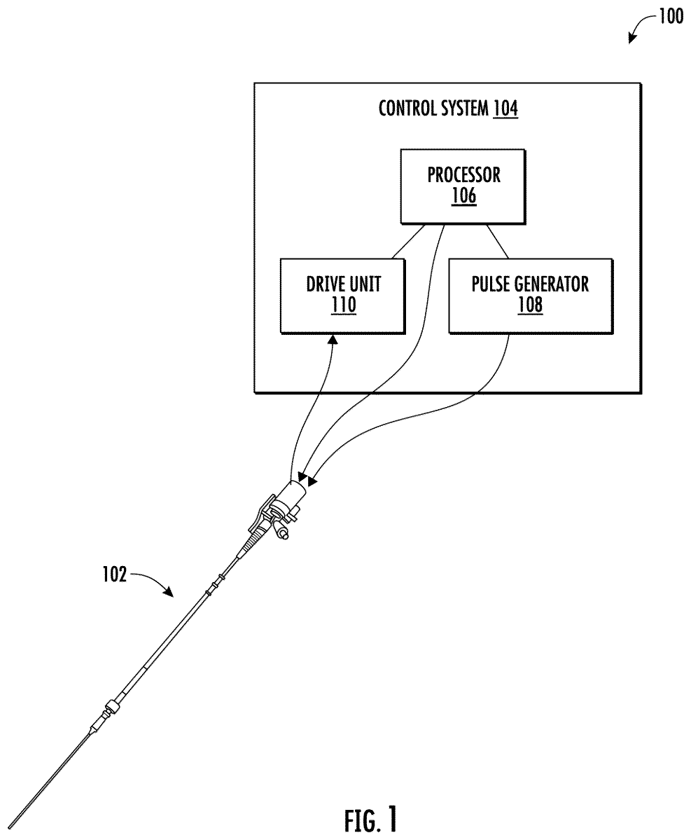

FIG. 1 illustrates schematically one embodiment of an IVUS imaging system 100. The IVUS imaging system 100 includes a catheter 102 that is couplable to a control system 104. The control system 104 may include, for example, a processor 106, a pulse generator 108, and a drive unit 110. In at least some embodiments, the pulse generator 108 forms electric pulses that may be input to one or more transducers (not shown) disposed in the catheter 102.

With some embodiments, mechanical energy from the drive unit 110 can be used to drive an imaging core (also not shown) disposed in the catheter 102. In at least some embodiments, electric signals transmitted from the one or more transducers may be input to the processor 106 for processing. In at least some embodiments, the processed electric signals from the one or more transducers can be used to form a series of images, described in more detail below. For example, a scan converter can be used to map scan line samples (e.g., radial scan line samples, or the like) to a two-dimensional Cartesian grid, which can be used as the basis for a series of IVUS images that can be displayed for a user.

In at least some embodiments, the processor 106 may also be used to control the functioning of one or more of the other components of the control system 104. For example, the processor 106 may be used to control at least one of the frequency or duration of the electrical pulses transmitted from the pulse generator 108, the rotation rate of the imaging core by the drive unit 110. Additionally, where IVUS imaging system 100 is configured for automatic pullback, the drive unit 110 can control the velocity and/or length of the pullback.

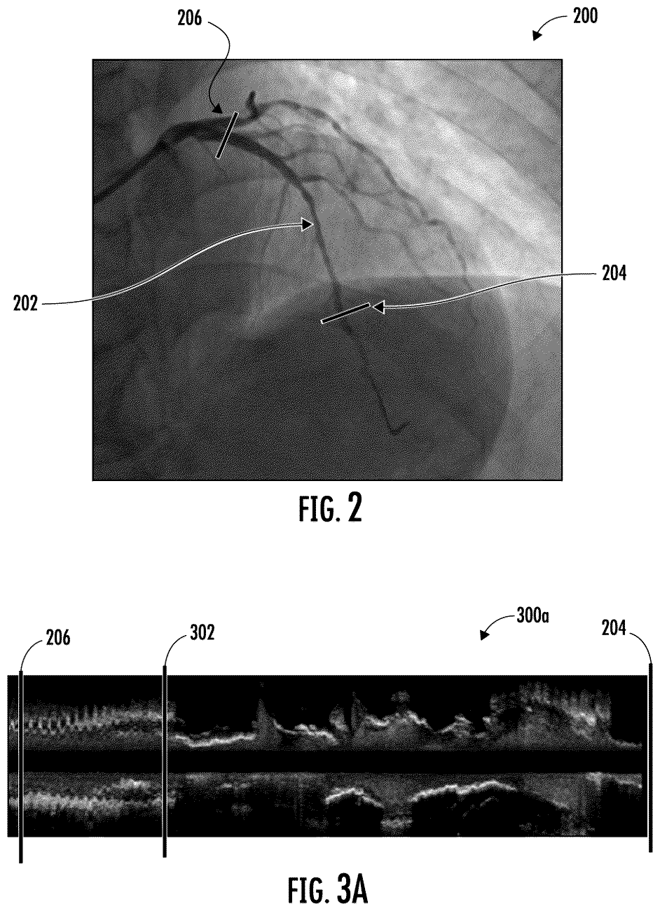

FIG. 2 illustrates an extravascular image 200 of a vessel 202 of a patient. As described, IVUS imaging systems (e.g., IVUS imaging system 100, or the like) are used to capture a series of intraluminal images or a “recording” or a vessel, such as, vessel 202. For example, an IVUS catheter (e.g., catheter 102) is inserted into vessel 202 and a recording, or a series of IVUS images, is captured as the catheter 102 is pulled back from a distal end 204 to a proximal end 206. The catheter 102 can be pulled back manually or automatically (e.g., under control of drive unit 110, or the like). The series of IVUS images captured between distal end 204 and proximal end 206 are often referred to an images from an IVUS run.

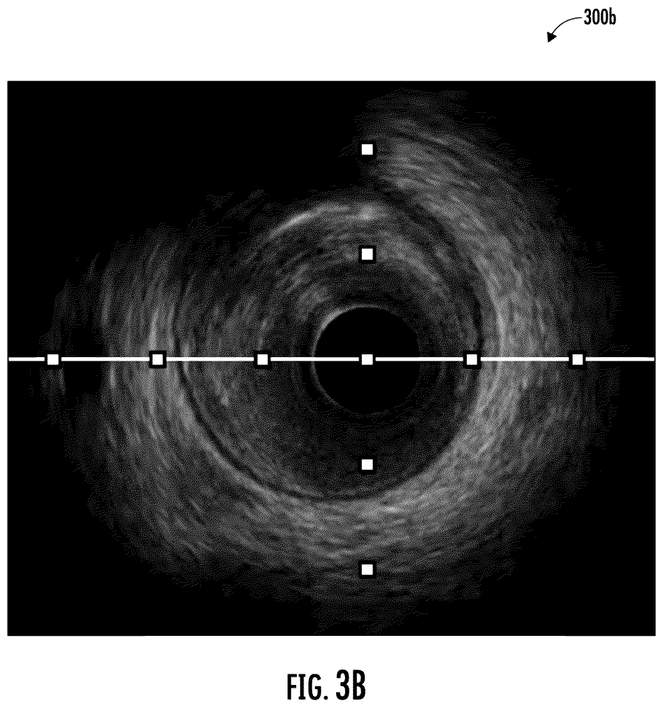

FIG. 3A and FIG. 3B illustrates two-dimensional (2D) representations of IVUS images of vessel 202. For example, FIG. 3A illustrates IVUS images 300a depicting a longitudinal view of the IVUS recording of vessel 202 between proximal end 206 and distal end 204.

FIG. 3B illustrates an image frame 300b depicting an on-axis (or short axis) view of vessel 202 at point 302. Said differently, image frame 300b is a single frame or single image from a series of IVUS images that can be captured between distal end 204 and proximal end 206 as described herein. As introduced above, a physician will often capture an IVUS run (e.g., series of IVUS images) at different stages of treatment. For example, IVUS images may be captured prior to a percutaneous coronary intervention (PCI) treatment and after the PCI treatment (e.g., placement of a stent, balloon dilation, rotablation, or the like) has been performed.

The present disclosure provides that the IVUS runs from different time frames can be aligned on a frame-by-frame basis and provide a graphical user interface that correlates the IVUS runs to allow the physician to view the correlated IVUS runs to observe a more direct understanding of their treatment on the vessel, for example, by observing the difference in lesion properties with a side-by-side comparison.

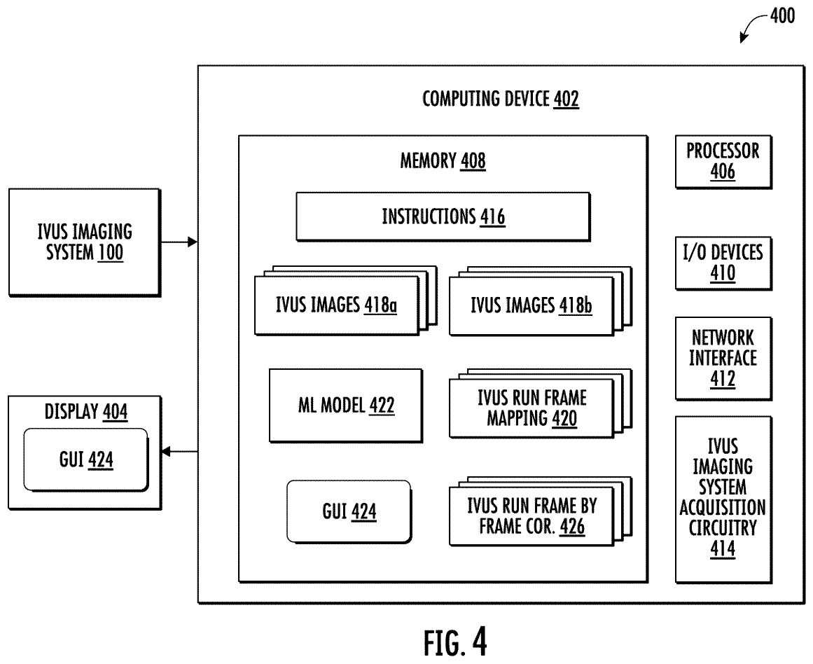

FIG. 4 illustrates an IVUS images correlation and visualization system 400, according to some embodiments of the present disclosure. In general, IVUS images correlation and visualization system 400 is a system for processing, correlating, and presenting multiple series of IVUS images of the same vessel. IVUS images correlation and visualization system 400 can be implemented in a commercial IVUS guidance or navigation system, such as, for example, the AVVIGO® Guidance System available from Boston Scientific®. The present disclosure provides advantages over prior or conventional IVUS navigation systems in that the no conventional systems provide to correlating IVUS runs taken at different times.

With some embodiments, IVUS images correlation and visualization system 400 could be implemented as part of control system 104. Alternatively, control system 104 could be implemented as part of IVUS images correlation and visualization system 400. As depicted, IVUS images correlation and visualization system 400 includes a computing device 402. Optionally, IVUS images correlation and visualization system 400 includes IVUS imaging system 100 and display 404.

It is noted that although the disclosure frequently uses IVUS as an exemplary intravascular imaging modality, the disclosure could be provided to longitudinally and/or angularly align frames from different runs captured using any of a variety of other intravascular imaging modalities, such as, optical coherence tomography (OCT).

Computing device 402 can be any of a variety of computing devices. In some embodiments, computing device 402 can be incorporated into and/or implemented by a console of display 404. With some embodiments, computing device 402 can be a workstation or server communicatively coupled to IVUS imaging system 100 and/or display 404. With still other embodiments, computing device 402 can be provided by a cloud based computing device, such as, by a computing as a service system accessibly over a network (e.g., the Internet, an intranet, a wide area network, or the like). Computing device 402 can include processor 406, memory 408, input and/or output (I/O) devices 410, network interface 412, and IVUS imaging system acquisition circuitry 414.

The processor 406 may include circuitry or processor logic, such as, for example, any of a variety of commercial processors. In some examples, processor 406 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 406 may include graphics processing portions and may include dedicated memory, multiple-threaded processing and/or some other parallel processing capability. In some examples, the processor 406 may be an application specific integrated circuit (ASIC) or a field programmable integrated circuit (FPGA).

The memory 408 may include logic, a portion of which includes arrays of integrated circuits, forming non-volatile memory to persistently store data or a combination of non-volatile memory and volatile memory. It is to be appreciated, that the memory 408 may be based on any of a variety of technologies. In particular, the arrays of integrated circuits included in memory 120 may be arranged to form one or more types of memory, such as, for example, dynamic random access memory (DRAM), NAND memory, NOR memory, or the like.

I/O devices 410 can be any of a variety of devices to receive input and/or provide output. For example, I/O devices 410 can include, a keyboard, a mouse, a joystick, a foot pedal, a display, a touch enabled display, a haptic feedback device, an LED, or the like.

Network interface 412 can include logic and/or features to support a communication interface. For example, network interface 412 may include one or more interfaces that operate according to various communication protocols or standards to communicate over direct or network communication links. Direct communications may occur via use of communication protocols or standards described in one or more industry standards (including progenies and variants). For example, network interface 412 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 412 can include logic and/or features to enable communication over a variety of wired or wireless network standards (e.g., 902.11 communication standards). For example, network interface 412 may be arranged to support wired communication protocols or standards, such as, Ethernet, or the like. As another example, network interface 412 may be arranged to support wireless communication protocols or standards, such as, for example, Wi-Fi, Bluetooth, ZigBee, LTE, 5G, or the like.

The IVUS imaging system acquisition circuitry 414 may include circuitry including custom manufactured or specially programmed circuitry configured to receive or receive and send signals between IVUS imaging system 100 including indications of an IVUS run, a series of IVUS images, or a frame or frames of IVUS images.

Memory 408 can include instructions 416. During operation processor 406 can execute instructions 416 to cause computing device 402 to receive (e.g., from IVUS imaging system 100, or the like) a series of IVUS images from multiple IVUS runs of a vessel and store the recording as IVUS images 418a, IVUS images 418b, etc. in memory 408. For example, processor 406 can execute instructions 416 to receive information elements from IVUS imaging system 100 comprising indications of IVUS images captured by catheter 102 while being pulled back from distal end 204 to proximal end 206, which images comprising indications of the anatomy and/or structure of vessel 202 including vessel walls and plaque. Further, it is to be appreciated that processor 406 can execute instructions 416 to receive IVUS images from multiple runs through a vessel (e.g., pre-PCI, post-PCI, at different times, or the like). It is to be appreciated that IVUS images 418a and 418b can be stored in a variety of image formats or even non-image formats or data structures that comprise indications of vessel 202. Further, IVUS images 418a and 418b includes several “frames” or individual images that, when represented co-linearly can be used to form an image of the vessel 202, such as, for example, as represented by IVUS images 300a.

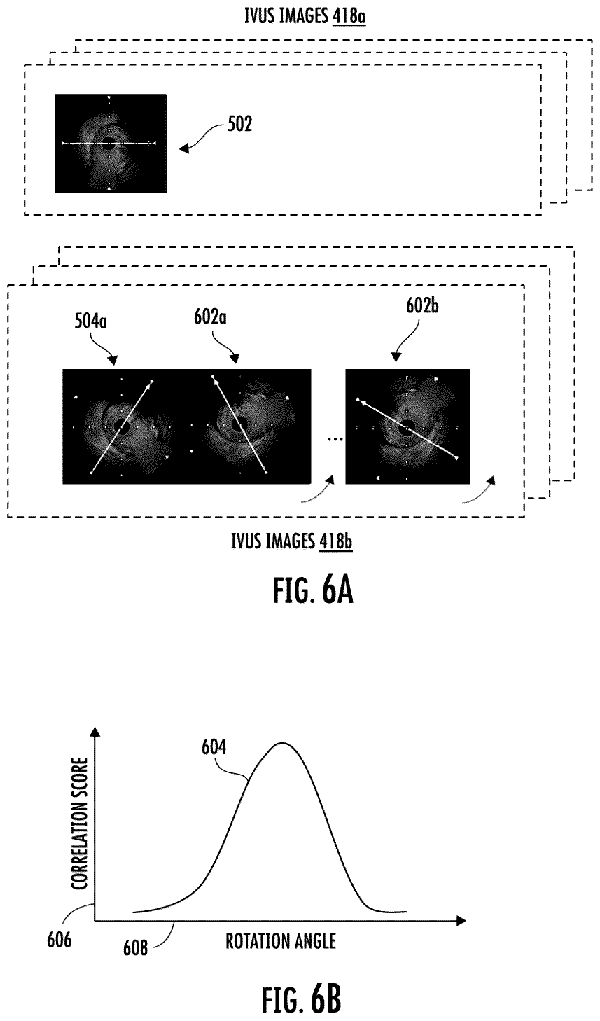

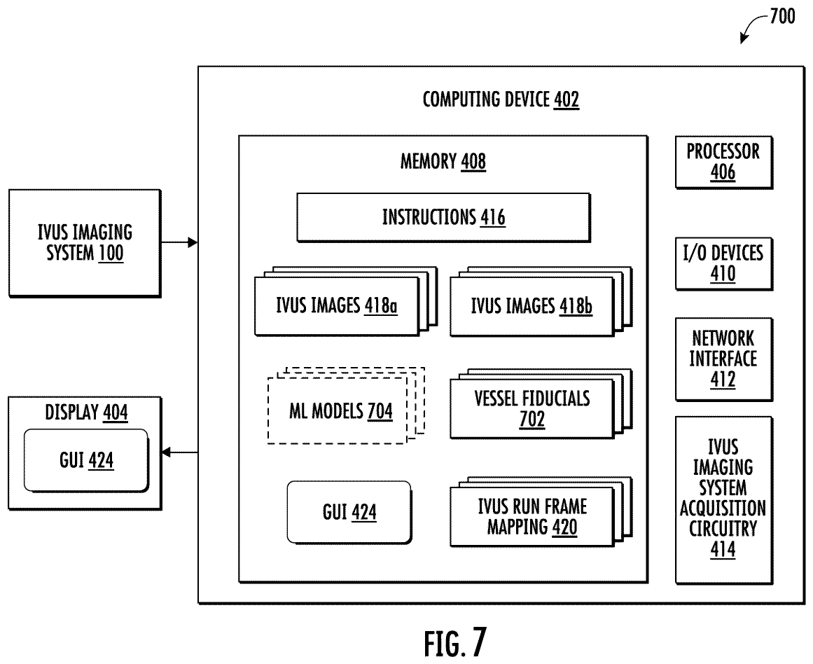

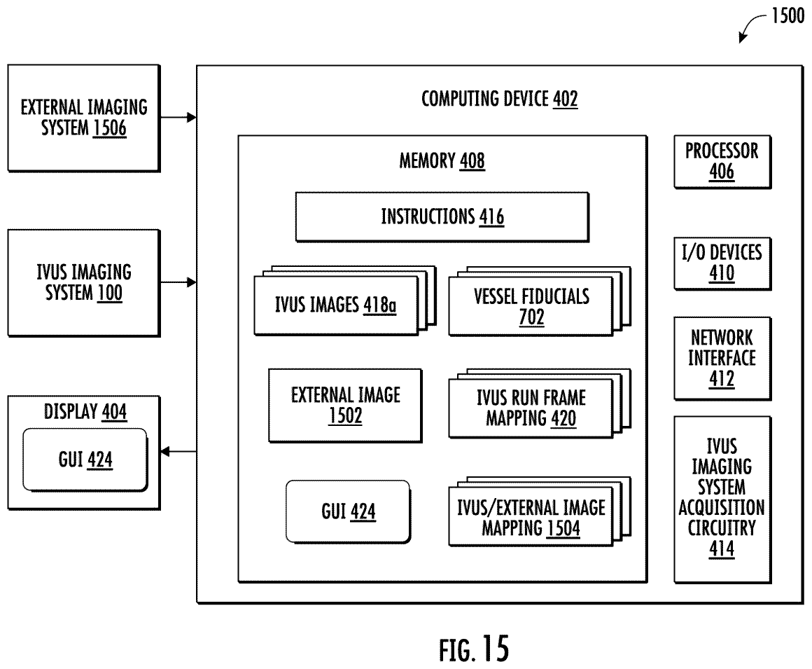

The present disclosure provides to correlate IVUS images 418a and IVUS images 418b on a frame-by-frame basis and present a correlated view of the images in a graphical user interface. With some examples, processor 406 can execute instructions 416 to identify IVUS run frame mapping 420 from IVUS images 418a and IVUS images 418b using a machine learning (ML) model to infer the mapping (e.g., see FIG. 4). With some embodiments, processor 406 can execute instructions 416 to identify IVUS run frame mapping 420 from IVUS images 418a and IVUS images 418b using a frame-by-frame correlation or a segment by segment correlation (e.g., see FIG. 4). With some embodiments, this can be facilitated using standard image processing techniques and/or ML inference. In other examples, memory 408 can execute instructions 416 to determine one or more fiducials (e.g., via machine learning, via image processing algorithms, or the like) and to determine IVUS run frame mapping 420 from the identified fiducials (e.g., see FIG. 7). Although each approach to determine the IVUS run frame mapping 420 is slightly different and discussed separately, the approaches are similar once the IVUS run frame mapping 420 is identified. Further, in some embodiments, an IVUS run can be mapped and/or aligned with an angiographic image of the vessel (e.g., see FIG. 6). It is important to note that FIG. 4, FIG. 7, and FIG. 15 depict IVUS images correlation and visualization systems, 400, 700, and 1500, respectively. This is done for clarity in describing the various alignment techniques disclosed herein. However, it is important to note that an alignment technique described with respect to one system (e.g., 400) can be used with an alignment technique disclosed with respect to another system (e.g., 700 and/or 1500). For example, it is to be appreciated that one alignment technique could be used to longitudinally align frames while another technique could be used to angularly align frames.

Turning now to FIG. 4. Once the IVUS run frame mapping 420 is generated, a frame-by-frame correlation between IVUS images 418a and IVUS images 418b can be generated from IVUS run frame mapping 420. For example, memory 408 can execute instructions 416 to correlate each frame of IVUS images 418a to a respective frame of IVUS images 418b. Further, memory 408 can execute instructions 416 to generate a graphical user interface (GUI) 424 depicting indications of frames of the IVUS images 418a correlated and/or with respect to respective frames of IVUS images 418b based on IVUS run frame mapping 420.

In the example where ML is used to generate IVUS run frame mapping 420, processor 406 can execute instructions 416 to execute or “run” ML model 422 with IVUS images 418a and IVUS images 418b as inputs to generate IVUS run frame mapping 420. ML model 422 can infer IVUS run frame mapping 420 from IVUS images 418a and IVUS images 418b. Memory 408 can store a copy of ML model 422 and processor 406 can execute ML model 422 to generate IVUS run frame mapping 420. In general, ML model 422 can be any of a variety of ML models. Examples of ML models and even training an ML model as contemplated herein are provided below.

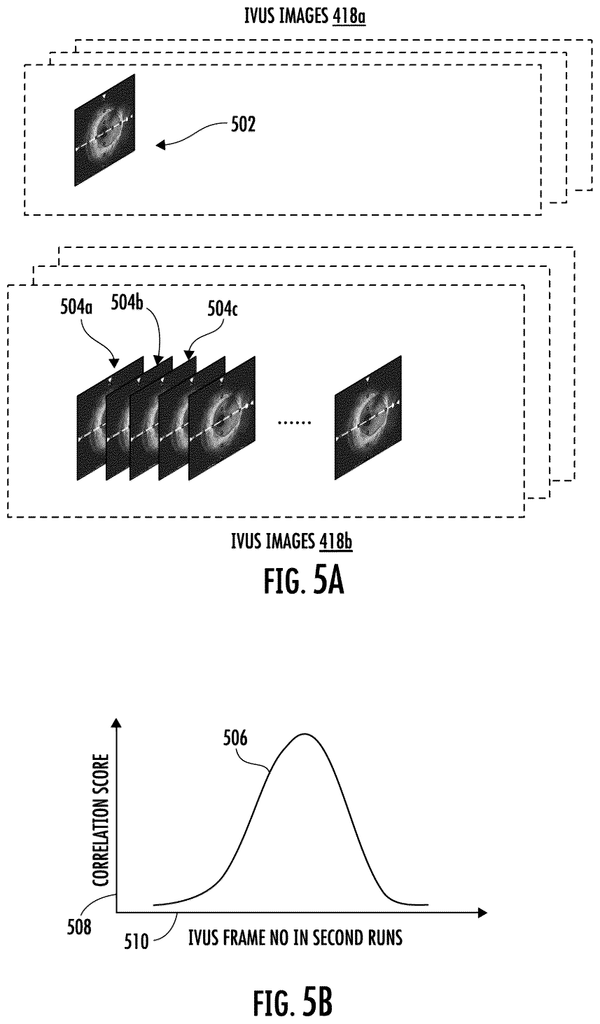

With some embodiments, the disclosure can be provided to align IVUS runs based on a correlation, for each frame of one IVUS run, with all frames of another IVUS run. Processor 406 could execute instructions 416 to determine an IVUS run frame-by-frame correlation 426. For examples, processor 406 could execute instructions 416 to iterate through each frame of IVUS images 418a and calculate (e.g., using fiducials, using ML, using background subtraction, using cross-correlation, or the like) the correlation between all frames of IVUS images 418b. Subsequently, processor 406 can execute instructions 416 to identify, for each frame in IVUS images 418a, the most closely correlated frame in IVUS images 418b. For example, FIG. 5A depicts an image frame 502 (e.g., from IVUS images 418a, or the like) and image frames 504a, 504b, 504c, etc. (e.g., from IVUS images 418b, or the like). Processor 406 can execute instructions 416 to calculate a correlation (e.g., correlation value, score, or the like) between the image frame 502 and the image frames 504a, 504b, 504c, etc. FIG. 5B illustrates a plot 506 of the calculated correlation. Plot 506 graphs the correlation score for a particular frame from one set of IVUS images (e.g., image frame 502) and the frames from another set of IVUS images (e.g., frames 504a, 504b, 504c, etc.) As can be seen, the value of the correlation score is plotted on the y axis 508 while the frame number from the second set of IVUS images is plotted on the x axis 510.

With some embodiments, the frame-by-frame correlation can be determined for each frame at different angles of rotation. Processor 406 can execute instructions 416 to identify, for each frame in a set of IVUS images (e.g., IVUS images 418a, or the like), a correlation with a frame from another set of IVUS images (e.g., IVUS images 418b, or the like) at several angles of rotation. FIG. 6A illustrates an example of this. During operation of IVUS images correlation and visualization system 400, processor 406 can execute instructions 416 to calculate a correlation score between frames from one set of IVUS images (e.g., image frame 502 from IVUS images 418a, or the like) and frames from another set of IVUS images (e.g., image frame 504a from IVUS images 418b, or the like) and rotated versions of the image frames (e.g., rotated image frames 602a and 602b). As with the frame-by-frame correlation described above, processor 406 can execute instructions 416 to calculate a correlation score between each frame from one set of IVUS images (e.g., IVUS images 418a) and each frame and rotated versions from another set of IVUS images (e.g., IVUS images 418b, or the like). FIG. 6B illustrates a plot 604 of the calculated correlation. Plot 604 graphs the correlation score for a particular image frame from one set of IVUS images (e.g., image frame 502) and an image frame from another set of IVU images (e.g., image frame 504a) and rotated versions that image frame (e.g., rotated image frames 602a and 602b). As can be seen, the value of the correlation score is plotted on the y axis 606 while the rotation angle is plotted on the x axis 608.

With some examples, processor 406 can execute instructions 416 to generate rotated image frames (e.g., rotated image frames 602a, 602b, etc.) at every possible angles of rotation. In such an example, 359 rotated image frames would be generated. In other examples, processor 406 can execute instructions 416 to generate rotated image frames at a subset of all possible angles of rotation (e.g., every 2 degrees, every 5 degrees, every 10 degrees, every 15 degrees, every 20 degrees, every 30 degrees, every 45 degrees, or the like).

In general, IVUS run frame mapping 420 can include an indication of an offset (e.g., in time, in distance, in rotation, or the like) in which to adjust one (or each) of the IVUS images 418a and IVUS images 418b to align them. As used herein, the term align is meant to align the frames of the images longitudinally and/or angularly.

With some embodiments, processor 406 can execute instructions 416 to receive a bookmark (or bookmarks) identifying a frame of one of IVUS images 418a and/or IVUS images 418b. The IVUS run frame mapping 420 can be adjusted to align the bookmark or bookmarks. With some embodiments, this mapping in not linear. For example, a frame from IVUS images 418a can be adjusted linearly (e.g., by a first distance) and/or rotated (e.g., by a first angle) based on its correlation to a frame from IVUS images 418b while the adjacent frame in IVUS images 418a can be adjusted linearly (e.g., by a second distance) and/or rotated (e.g., by a second angle) based on its correlation to the same or a different frame from IVUS images 418b.

FIG. 7 illustrates an IVUS images correlation and visualization system 700, according to some embodiments of the present disclosure. In general, IVUS images correlation and visualization system 700 is a system for processing, correlating, and presenting multiple series of IVUS images of the same vessel, similarly to IVUS images correlation and visualization system 400. To simplify the discussion, many of the components of IVUS images correlation and visualization system 400 are referenced in and reused in IVUS images correlation and visualization system 700.

As described above with respect to FIG. 4 and IVUS images correlation and visualization system 400, the disclosure provides to generate IVUS run frame mapping 420 for IVUS images 418a and IVUS images 418b. In some embodiments, processor 406 can execute instructions 416 to identify vessel fiducials 702 in IVUS images 418a and IVUS images 418b. With some embodiments, vessel fiducials 702 can be any one or more coronary anatomical fiducials (e.g., lumen geometry, vessel geometry, side branch locations, calcium morphology, plaque distribution, guide catheter position, or the like). In some embodiments, processor 406 executes instructions 416 to identify vessel fiducials 702 from IVUS images 418a and IVUS images 418b using image processing algorithms (e.g., geometric image identification algorithms to identify lumen profile, or the like). In other embodiments, memory 408 can include one or more ML models 704 configured to infer a vessel fiducials 702 from IVUS images (e.g., IVUS images 418a, 418b, etc.). For example, memory 408 can include ML models 704, where ML models 704 can include one or more ML models trained to infer a fiducial (e.g., a side branch location, a calcium morphology, a guide catheter position, or the like). As such, processor 406 can execute ML models 704 to identify vessel fiducials 702 in frames of IVUS images 418a and IVUS images 418b.

Processor 406 can execute instructions 416 to generate IVUS run frame mapping 420 from vessel fiducials 702, for example, by pairing frames from IVUS images 418a and IVUS images 418b where the same anatomical fiducial was identified. Given IVUS run frame mapping 420, processor 406 can execute instructions 416 to correlate each frame of IVUS images 418a to a respective frame of IVUS images 418b. Further, processor 406 can execute instructions 416 to generate GUI 424 depicting indications of frames of the IVUS images 418a correlated and/or with respect to respective frames of IVUS images 418b based on IVUS run frame mapping 420.

It is to be appreciated that in some embodiments, processor 406 can execute instructions 416 and identify a fiducial in a single frame of each IVUS run (e.g., IVUS images 418a and 418b). For example, vessel fiducials 702 could include a side branch identified in a frame of IVUS images 418a and the same side branch identified in a frame of IVUS images 418b. In other embodiments, processor 406 can execute instructions 416 and identify fiducials in multiple frames. In such an example, the fiducials need not be the same. For example, like stated above, vessel fiducials 702 could include a side branch location in a frame of IVUS images 418a and the same side branch location in a frame of IVUS images 418b as well as a guide catheter location in another frame of IVUS images 418a and the guide catheter location in another frame of IVUS images 418b. Examples are not limited in this context.

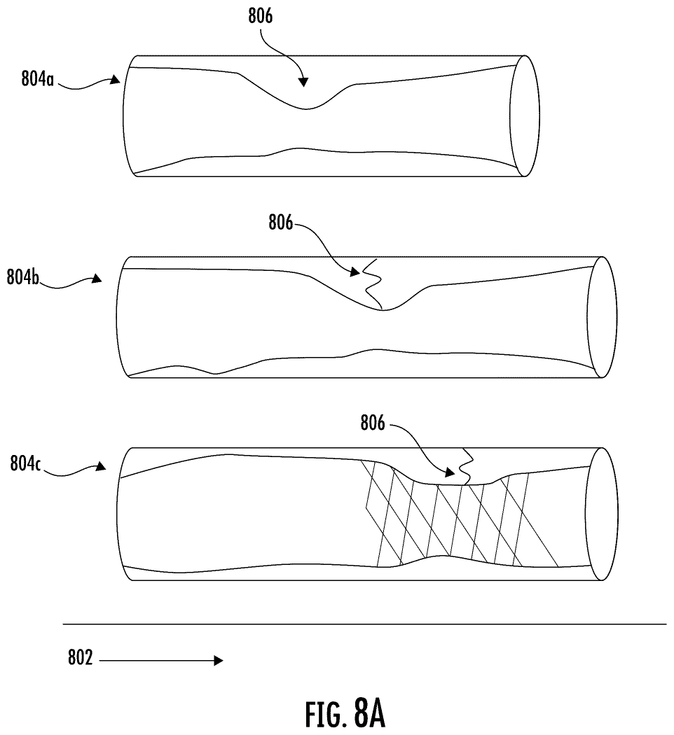

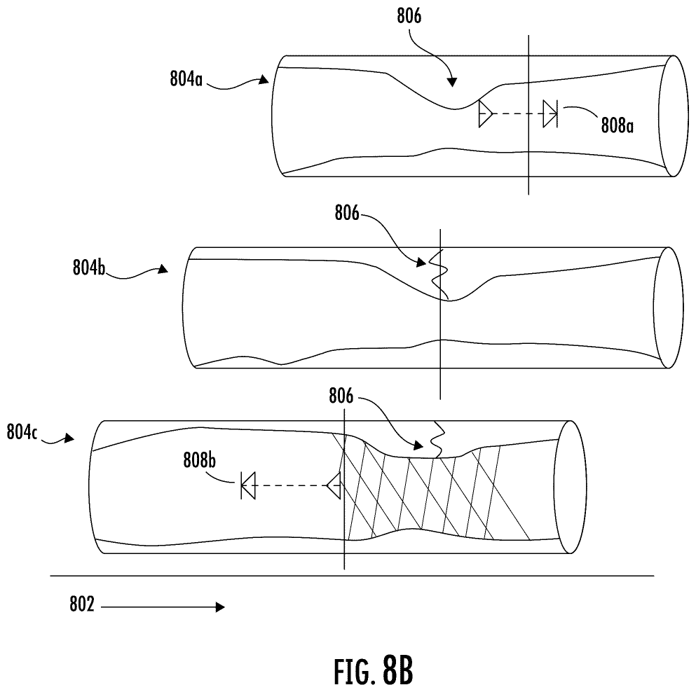

FIG. 8A illustrates several IVUS runs set against scale 802. IVUS runs, or sets of IVUS images 804a, 804b, and 804c are depicted in this figure. It is to be appreciated that each set of IVUS images (e.g., sets of IVUS image 804a, 804b, and 804c) includes several frames. As outlined above, with some embodiments, a fiducial is identified in one or more frames of each set of IVUS images. This figure depicts fiducial 806 identified in a frame of each set of IVUS images 804a, 804b, and 804c. IVUS run frame mapping 420 can be generated from the frames identified as indicating (representing, corresponding to, depicting, or the like) fiducial 806. For example, processor 406 can execute instructions 416 to identify a frame from each set of IVUS images (e.g., a frame from images 804a, images 804b, and images 804c, or the like) comprising the fiducial 806 of the vessel (or vessel fiducial). Processor 406 can execute instructions 416 to identify an offset to frames of a set (or multiple sets) of the IVUS images, which when applied will align the frames in each set of IVUS images on the scale 802. In some embodiments, the offset can be a time offset, a distance offset, an angle offset, or any combination of a time, distance and/or angle offset. Further, it is to be appreciated that the scale 802 can be any scale in which the IVUS run is represented or graphically presented. For example, some IVUS runs are graphically represented against a pullback scale with distal and proximal points along the pullback. As an example, pullback scale can be represented in a unit of distance (e.g., millimeters, or the like). In the context of an offset, the offset can be generated such that the frames identified as indicating the same fiducial (e.g., vessel fiducial 806) are shifted or adjusted when the offset is applied such that the frames are aligned on the scale.

For example, FIG. 8B illustrates the IVUS images from FIG. 8A again set against scale 802. However, the frames from the sets of IVUS images 804a and 804c have been adjusted based on identified offsets (e.g., from IVUS run frame mapping 420, or the like) to align the frames indicating the fiducial on the scale 802. For example, IVUS images 804a are adjusted an offset 808a to shift the IVUS images 804a with respect to the scale 802 while IVUS images 804c are adjusted an offset 808b to shift the IVUS images 804c with respect to scale 802. Applying the offsets 808a and 808b to the IVUS images 804a and 804c, respectively, aligns the IVUS images against scale 802 and particularly aligns the frames in IVUS images 804a, 804b, and 804c that indicate the vessel fiducial 806 against the scale 802. For example, as depicted in this figure, the identified fiducial 806 in each IVUS run aligns when the IVUS image 804a, 804b, and 804c are adjusted based on offsets 808a and 808b. It is noted that the offsets 808a and 808b are depicted as a longitudinal offset, or rather a distance to offset the frames along scale 802. However, the offsets 808a and 808b could instead by an offset angle (e.g., an angle with which to rotate the frames) or could be both an offset distance and an offset angle. Further, it is noted that although only a single offset per IVUS run is depicted (e.g., offset 808a for IVUS images 804a and offset 808b for IVUS images 804c) multiple offsets for each run (e.g., for frames in a segment, for each frame, for only some of the frames, etc.) could be provided herein.

A variety of techniques and workflows to identify longitudinal and/or angular offsets for frames in a set of IVUS images (e.g., IVUS images 418a, or the like) to align the frames with frames in another set of IVUS images (e.g., IVUS images 418b, or the like) are provided. It is noted that although FIG. 8A and FIG. 8B depict longitudinal alignment only, the disclosure can be implemented to align IVUS runs longitudinally, angularly, and/or longitudinally and angularly.

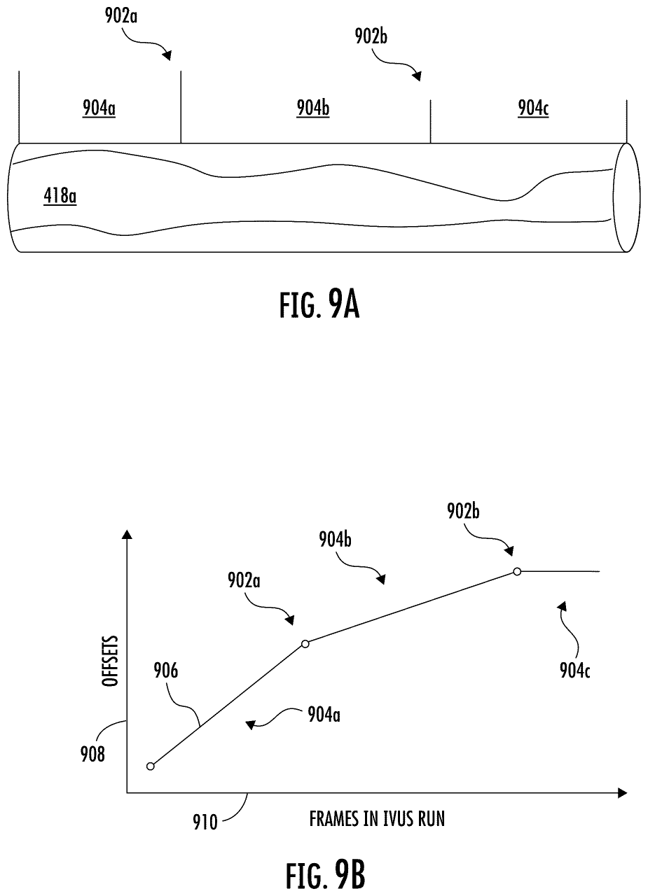

With some embodiments, processor 406 can execute instructions 416 to longitudinally align frames from IVUS images 418a with frames from IVUS images 418b on a segment by segment basis. For example, with some embodiments, processor 406 can execute instructions 416 to identify segments based on vessel fiducials 702. FIG. 9A illustrates IVUS images 418a and identified fiducials 902a and 902b. As outlined above, these fiducials can be side branches, lumen geometry, vessel geometry, calcium morphology, plaque distribution, etc. Processor 406 can execute instructions 416 to group frames from IVUS images 418a into segments based on the identified fiducials 902a and 902b. For example, FIG. 9A illustrates frames from IVUS images 418a grouped into segments 904a, 904b, and 904c. Accordingly, an offset for frames in a set of IVUS images (e.g., IVUS images 418a, or the like) can be generated for different segments using the identified vessel fiducials 702.

FIG. 9B illustrates points representing each longitudinal offset for frames corresponding to fiducials 902a and 902b. From these points, a plot 906 can be generated representing longitudinal offsets, plotted on the y axis 908, for each frame in IVUS run 418a, plotted on the x axis 910. With some embodiments, plot 906 can be generated linearly between the points (e.g., as depicted in FIG. 9B). In other embodiments, processor 406 can execute instructions 416 to generate plot 906 based on one or more line fitting algorithms (e.g., raster based line fitting, etc.). The longitudinal offset for frames in each segment 904a, 904b, and 904c can be determined based on the plot 906.

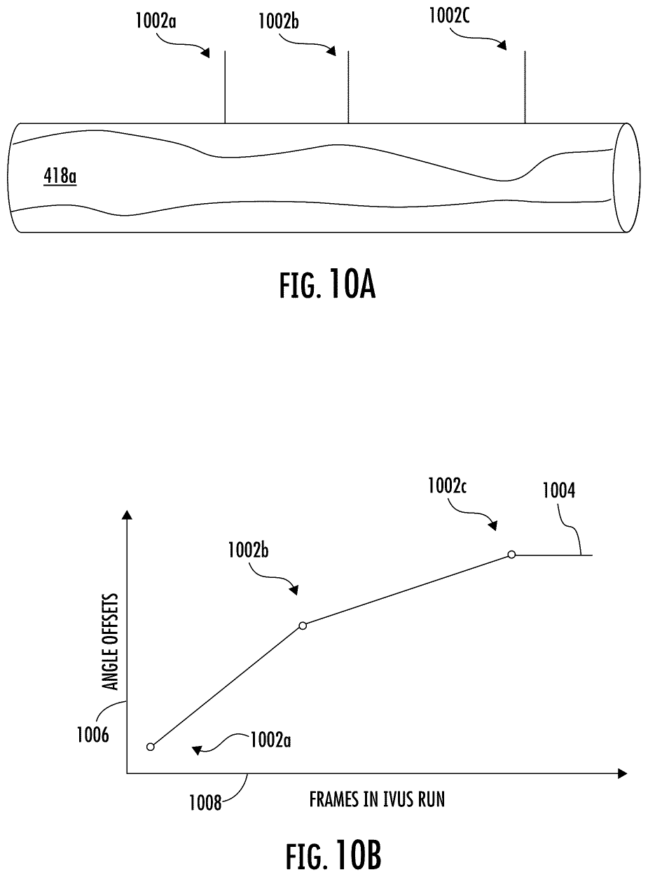

With some embodiments, processor 406 can execute instructions 416 to rotationally align frames from IVUS images 418a with frames from IVUS images 418b based on vessel fiducials 702. For example, in some embodiments, the IVUS run frame mapping 420 can include an offset angle (e.g., with which to rotate the frame). FIG. 10A illustrates IVUS images 418a and identified fiducials 1002a, 1002b, and 1002c. As outlined above, these fiducials can be side branches, lumen geometry, vessel geometry, calcium morphology, plaque distribution, etc. As outlined above, frames in IVUS images 418a corresponding to fiducials 1002a, 1002b, and 1002c can be mapped to a particular frame in IVUS images 418b (e.g., based on vessel fiducials 702, or the like) and an offset angle between the frames can be determined. In another embodiments, an offset angle can be determined based on calculating a correlation with each frame and rotated versions of each frame (e.g., as described above with respect to FIG. 6A, and FIG. 6B).

FIG. 10B illustrates points representing each offset angle for frames corresponding to fiducials 1002a, 1002b, and 1002c. From these points, a plot 1004 can be generated representing offset angles, plotted on the y axis 1006, for each frame in IVUS run 418a, plotted on the x axis 1008. As noted above, the 1004 can be generated linearly and/or based on one or more line fitting or line smoothing algorithms.

It is to be appreciated that various techniques and workflows to identify an alignment offset can be combined. As used herein, “alignment offset” is intended to mean either an offset distance (e.g., to longitudinally align frames) or an offset angle (e.g., to angularly align frames), or both. For example, IVUS run frame mapping 420 can include either or both an offset distance and an offset angle. With some examples, various offset derivation methodologies outlined herein can be combined in a segment-by-segment basis. For example, an alignment offset for frames in a first segment (e.g., segment 904a of FIG. 9A, or the like) can be determined based on a first selection of alignment methodologies disclosed herein while an alignment offset for frames in another segment (e.g., segment 904b, 904c, etc. of FIG. 9A) can be determined based on a second selection of alignment methodologies disclosed herein. As a specific example, frames in segment 904a can be aligned using a frame-by-frame correlation while frames in segment 904b can be aligned using inference from an ML model. Claims, however, are not limited to just this example but can include any combination of techniques implemented on a segment-by-segment basis.

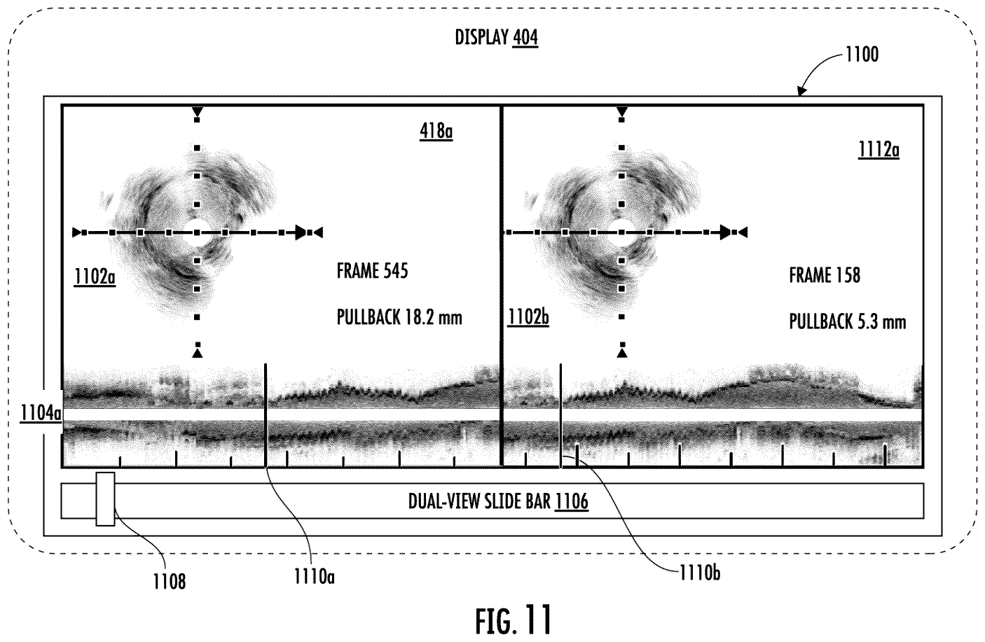

As discussed above, a GUI can be generated to present graphical indications of the different IVUS runs in relation to each other, such as for example, where the frames are aligned as described herein. FIG. 11 illustrates a GUI 1100, which can be generated in accordance with some embodiments of the present disclosure. In some embodiments, GUI 1100 can be GUI 424 of FIG. 4, FIG. 7, or FIG. 15. For example, processor 406 can execute instructions 416 to generate GUI 424 having graphical components and an arrangement as depicted in GUI 1100 of FIG. 11. In such an example, processor 406 can execute instructions 416 to cause GUI 1100 to be displayed on display 404.

GUI 1100 can include graphical indications of IVUS images 418a and IVUS images 418b. As shown in this example, graphical indication of IVUS images 418a and IVUS images 418b include both an on-axis view (e.g., on-axis view 1102a and on-axis view 1102b) and a longitudinal view (e.g., longitudinal view 1104a and longitudinal view 1104b). As depicted GUI 1100 can arrange the on-axis view 1102a and the on-axis view 1102b as well as longitudinal view 1104a and longitudinal view 1104b in a horizontal (e.g., side-by-side) visualization. With other embodiments, processor 406 can execute instructions 416 to generate GUI 1100 to visualize the on-axis view 1102a and the on-axis view 1102b in a vertical arrangement.

Further, GUI 1100 can include a dual-view slide bar 1106 and a dual-view slider 1108. The dual-view slider 1108 can be manipulated (e.g., via a touch screen, via a mouse, via a joystick, or the like) to slide (or move) through the frames of the IVUS images. As dual-view slider 1108 is moved, processor 406 can execute instructions 416 to regenerate GUI 1100 to move frame indicators 1110a and 1110b disposed over longitudinal views 1104a and 1104b along with the position of the dual-view slider 1108. Further still, the on-axis views 1102a and 1102b can change to correspond to the frames from each respective IVUS run matching the location of the frame indicators 1110a and 1110b.

Accordingly, as provided herein, one or both IVUS runs can be adjusted (e.g., based on an offset distance and/or an offset angle) to align the IVUS runs with each other. As such, a user (e.g., physician) can view different IVUS runs (e.g., a pre-PCI run and a post-PCI run, or the like) where the locations, and corresponding fiducials, of the vessel are aligned in the visualization, such as for example, as depicted in GUI 1100.

With some embodiments, more than two (2) IVUS runs can be presented in a GUI. For example, FIG. 8A and FIG. 8B show three (3) IVUS runs that are shifted to align the IVUS runs with each other. Accordingly, GUI 1100 could be generated to present graphical indications for each of these three (3) IVUS runs.

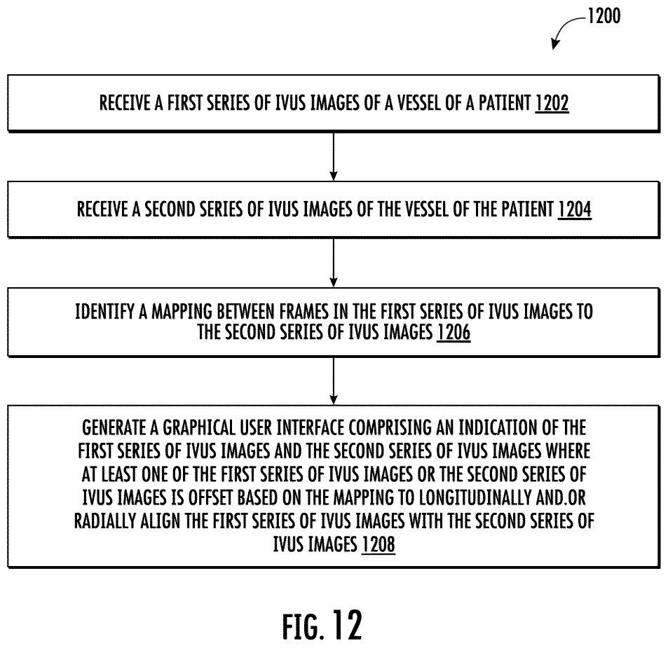

FIG. 12 illustrates a logic flow 1200 to align different IVUS runs, according to some embodiments of the present disclosure. The logic flow 1200 can be implemented by an IVUS images correlation and visualization system described herein, such as for example, IVUS images correlation and visualization system 400, 700, etc. For clarity and not limitation, the logic flow 1200 is described with reference to IVUS images correlation and visualization system 400.

Logic flow 1200 can begin at block 1202. At block 1202 “receive a first series of IVUS images of a vessel of a patient” a first series of IVUS images captured via an IVUS catheter percutaneously inserted in a vessel of a patent can be received. For example, information elements comprising indications of IVUS images 418a can be received from IVUS imaging system 100 where catheter 102 is (or was) percutaneously inserted into vessel 202. The IVUS images 418a can comprise frames of images representative of images captured while the catheter 102 is pulled back from distal end 204 to proximal end 206. Processor 406 can execute instructions 416 to receive information elements comprising indications of IVUS images 418a from IVUS imaging system 100, or directly from catheter 102 as may be the case.

Continuing to block 1204 “receive a second series of IVUS images of the vessel of the patient” a second series of IVUS images captured via an IVUS catheter percutaneously inserted in the vessel of the patent can be received. For example, information elements comprising indications of IVUS images 418b can be received from IVUS imaging system 100 where catheter 102 is (or was) percutaneously inserted into vessel 202. Like IVUS images 418a, IVUS images 418b can comprise frames of images representative of images captured while the catheter 102 is pulled back from distal end 204 to proximal end 206. However, as described above and contemplated herein, distal end 204 and proximal end 206 for IVUS images 418a can be at different locations than distal end 204 and proximal end 206 for IVUS images 418b. Processor 406 can execute instructions 416 to receive information elements comprising indications of IVUS images 418b from IVUS imaging system 100, or directly from catheter 102 as may be the case.

Continuing to block 1206 “identify a mapping between frames in the first series of IVUS images to the second series of IVUS images” a mapping between frames in the first series of IVUS images to frames in the second series of IVUS images can be identified. For example, processor 406 can execute instructions 416 to generate IVUS run frame mapping 420 based on ML model 422. In another embodiments, processor 406 can execute ML models 704 to identify vessel fiducials 702 and then identify IVUS run frame mapping 420 from vessel fiducials 702. In another example, processor 406 can execute instructions 416 to generate IVUS run frame mapping 420 on based on a correlation (e.g., frame-by-frame correlation, angular offset frame-by-frame correlation, or the like) as outlined above. In yet another example, processor 406 can execute instructions 416 to generate IVUS run frame mapping 420 on a per segment basis as outlined above.

In any of the above embodiments, IVUS run frame mapping 420 can comprise an indication of an offset (e.g., in time, in distance, in angle, or the like) for one or both series of IVUS images, which when applied would align the IVUS images longitudinally (e.g., as depicted in FIG. 8B) and/or angularly. As described herein, the IVUS run frame mapping 420 can indicate offset distances and/or offset angles. Examples are not limited in this context.

With some examples, processor 406 can execute instructions 416 to map frames based on a longitudinal offset as outlined herein. In such an example, processor 406 can execute instructions 416 to map frames based on a partial overlap and time warping. It is to be appreciated that one set of IVUS images (e.g., IVUS images 418a, or the like) can be captured at a first pullback speed while another set of IVUS images (e.g., IVUS images 418b, or the like) can be captured at a second pullback speed, which is different from the first pullback speed. With yet another example, one set of IVUS images (e.g., IVUS images 418a, or the like) can be captured along a first pullback path through a vessel while another set of IVUS images (e.g., IVUS images 418b, or the like) can be captured along a slightly different pullback path, or motion artifacts can be manifest in the captured IVUS images.

Accordingly, although many of the examples discuss aligning (or co-registering) IVUS images of different runs based on offset distances and/or angles, some embodiments provide that the runs can also be aligned (or co-registered) based on motion overlaps and/or time warping.

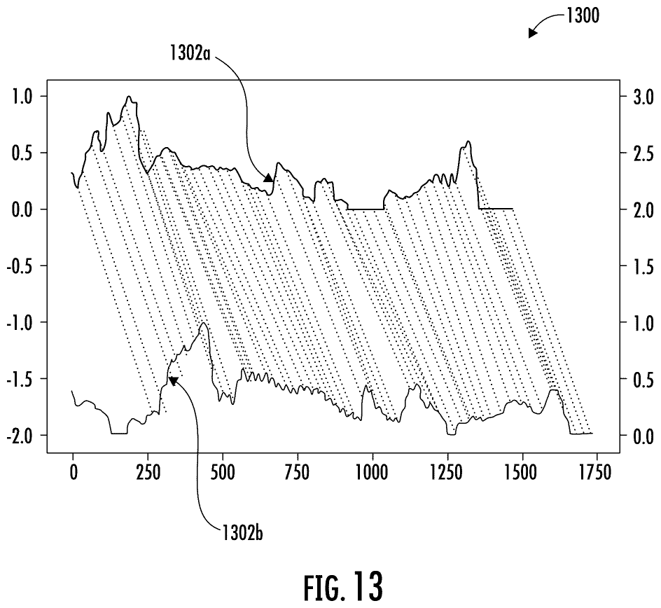

For example, FIG. 13 illustrates a plot 1300 showing alignment of extracted and vectorized features from two IVUS runs through a vessel. Extracted and vectorized features 1302a could be generated from IVUS images 418a while extracted and vectorized features 1302b could be generated from IVUS images 418b. These features can be aligned based on time-warping along the longitudinal offset as discussed herein. That is, as depicted in this figure, the frames of the IVUS runs can be shifted different amounts longitudinally to account for varying pullback speeds and paths through the vessel.

Continuing to block 1208 “generate a graphical user interface comprising an indication of the first series of IVUS images and the second series of IVUS images where at least one of the first series of IVUS images or the second series of IVUS images is offset (e.g., in time, in distance, in angle, or the like) based on the mapping to longitudinally and/or angularly align the first series of IVUS images with the second series of IVUS images” a GUI can be generated where the GUI comprises graphical indications of the first series of IVUS images and the second series of IVUS images and where any number of frames from the first and/or second series of IVUS images is offset (e.g., in time, in distance, in angle, or the like) to longitudinally and/or angularly align the first and second series of IVUS images. For example, processor 406 can execute instructions 416 to generate GUI 424 as discussed above. As a specific example, processor 406 can execute instructions 416 to generate GUI 1100 as GUI 424 and cause GUI 1100 to be displayed on display 404.

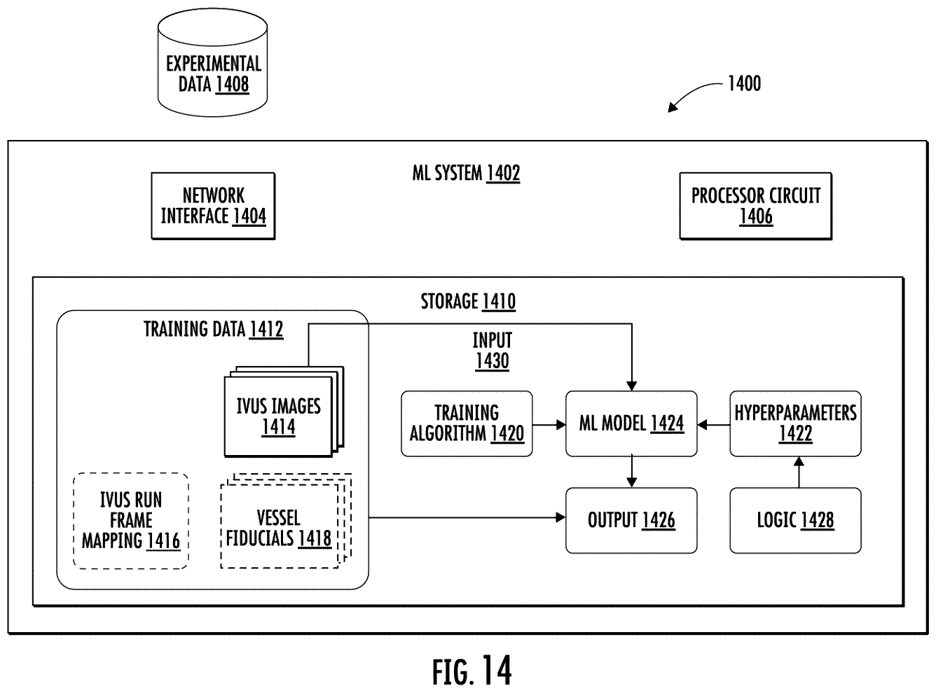

As noted, with some embodiments, processor 406 of computing device 402 can execute instructions 416 to generate IVUS run frame mapping 420 using an ML model or to generate vessel fiducials 702 from an ML model and then generate IVUS run frame mapping 420 from vessel fiducials 702. In such examples, the ML model can be stored in memory 408 of computing device 402. It will be appreciated, that prior to being deployed, the ML model is to be trained. FIG. 14 illustrates an ML environment 1400, which can be used to train an ML model that may later be used to generate (or infer) a mapping or vessel fiducials as outlined herein. The ML environment 1400 may include an ML system 1402, such as a computing device that applies an ML algorithm to learn relationships between an input and an inferred output. In this example, the ML algorithm can learn relationships between an input (e.g., IVUS images) and an output (e.g., a frame mapping or vessel fiducials depending on the embodiment).

The ML system 1402 may make use of experimental data 1408 gathered during several prior procedures. Experimental data 1408 can include IVUS images from several IVUS runs for several patients. The experimental data 1408 may be collocated with the ML system 1402 (e.g., stored in a storage 1410 of the ML system 1402), may be remote from the ML system 1402 and accessed via a network interface 1504, or may be a combination of local and remote data.

Experimental data 1408 can be used to form training data 1412. As noted above, the ML system 1402 may include a storage 1410, which may include a hard drive, solid state storage, and/or random access memory. The storage 1410 may hold training data 1412. In general, training data 1412 can include information elements or data structures comprising indications of multiple series of IVUS images and corresponding desired output (e.g., either a mapping or vessel fiducials). It is to be appreciated that where the desired output is an IVUS frame mapping then the input can be two (or more as may be the case) series of IVUS images. As a specific example referring to FIG. 4, where ML model 1424 is to be trained and deployed as ML model 422, the input can be multiple pairs of a first series of IVUS images and second series of IVUS images (e.g., more than one IVUS run) and the output can be a mapping associated with each pair of first and second series of IVUS images (e.g., mapping between the IVUS runs). In another example, referring to FIG. 7, where ML model 1424 is to be trained and deployed as ML models 704, the input can be a single series of IVUS images (e.g., a single IVUS run) and the output can be frames in the IVUS images where a vessel fiducial (or fiducials) is identified.

The training data 1412 may be applied to train the ML model 1424. Depending on the application, different types of models may be used to form the basis of ML model 1424. For instance, in the present example, an artificial neural network (ANN) may be particularly well-suited to learning associations between an IVUS images (e.g., IVUS images 418a, IVUS images 418b, etc.) and fiducials or frame mapping (e.g., IVUS run frame mapping 420, vessel fiducials 702, etc.) Convoluted neural networks may also be well-suited to this task. In another example, ML model 1424 can be based on a spatial transformer (e.g., a spatial transformation network, or the like). As another example, ML model 1424 can be multiple networks, such as, for example, Siamese networks, or the like.

Any suitable training algorithm 1420 may be used to train the ML model 1424. For example, the examples depicted herein may be suited to a supervised training algorithm or reinforcement learning training algorithm. For a supervised training algorithm, the ML system 1402 may apply the IVUS images 1414 as inputs 1430, to which an expected output (e.g., mapping or fiducials) can be generated by ML model 1424. In a reinforcement learning scenario, training algorithm 1420 may attempt to maximize some or all (or a weighted combination) of the model inputs 1430 mappings to output 1426 to produce an ML model 1424 having the least error. With some embodiments, training data 1412 can be split into “training” and “testing” data wherein some subset of the training data 1412 can be used to adjust the ML model 1424 (e.g., internal weights of the model, or the like) while another, non-overlapping subset of the training data 1412 can be used to measure an accuracy of the ML model 1424 to infer (or generalize) output 1426 from “unseen” input 1430.

The ML model 1424 may be applied using a processor circuit 1406, which may include suitable hardware processing resources that operate on the logic and structures in the storage 1410. The training algorithm 1420 and/or the development of the trained ML model 1424 may be at least partially dependent on hyperparameters 1422. In exemplary embodiments, the model hyperparameters 1422 may be automatically selected based on logic 1428, which may include any known hyperparameter optimization techniques as appropriate to the ML model 1424 selected and the training algorithm 1420 to be used. In optional, embodiments, the ML model 1424 may be re-trained over time, to accommodate new knowledge and/or updated experimental data 1424.

Once the ML model 1424 is trained, it may be applied (e.g., by the processor 406, or the like) to new input data (e.g., IVUS images 418a, IVUS images 418b, etc.) This input to the ML model (e.g., ML model 422, ML model 702, or the like) may be formatted according to a predefined model inputs 1430 mirroring the way that the training data 1412 was provided to the ML model 1424. The ML model 1424 may generate output 1426 which may be, for example, a generalization or IVUS run frame mapping 420 or vessel fiducials 702 as discussed above.

The above description pertains to a particular kind of ML system 1402, which applies supervised learning techniques given available training data with input/output pairs. However, the present invention is not limited to use with a specific ML paradigm, and other types of ML techniques may be used. For example, in some embodiments the ML system 1402 may apply for example, evolutionary algorithms, or other types of ML algorithms and models to an IVUS run frame mapping 420 (or vessel fiducials 702 as may be the case) from IVUS images 418a and/or IVUS images 418b.

With some embodiments, ML model 1424 can be a traditional ML model, such as, for example, a neural network, a convolutional neural network, an evolutionary artificial neural network, or the like. However, in some embodiments, ML model 1424 may not be an ML model in the traditional sense. For example, ML model 1424 might be a dynamic programming algorithm where parameters of the dynamic programming algorithm are tuned using the training data 1412.

In some embodiments, the disclosure can be provided to angularly align an IVUS run with a view of the vessel from an external imaging modality. For example, FIG. 15 illustrates an IVUS images correlation and visualization system 1500, according to some embodiments of the present disclosure. In general, IVUS images correlation and visualization system 1500 is a system for processing, correlating, and presenting IVUS images with an external image of the same vessel. To simplify the discussion, many of the components of IVUS images correlation and visualization system 400 are referenced in and reused in describing IVUS images correlation and visualization system 1500.

As described above with respect to FIG. 4 and IVUS images correlation and visualization system 400, the disclosure provides to generate IVUS run frame mapping 420 for IVUS images 418a and IVUS images 418b. In some embodiments, IVUS run frame mapping 420 can be generated based on an external image of the vessel. It is noted that a variety of techniques exist to co-register intravascular images (e.g., IVUS images 418a and/or 418b) with an external image. The present disclosure does not reproduce such techniques herein. However, for clarity, it is noted that fiducials can be identified on an external image like on an intravascular image and the fiducial mapped to each other to cp-register frames in the intravascular images to points (e.g., in x and y coordinates) on the external image.

As such, with some examples, IVUS images correlation and visualization system 1500 can be coupled to an external imaging system 1506 (e.g., an angiography machine, a computed tomography (CT) machine, a magnetic resonant imaging (MRI) machine, or the like) that is configured to capture external images of the vessel with which IVUS images 418a and/or 418b are captured. Alternatively, IVUS images correlation and visualization system 1500 can be coupled to a memory device storing external images or frames of external images.

Processor 406 can execute instructions 416 to receive an external image 1502 (or images) from external imaging system 1506 (or a memory storage device). Processor 406 can execute instructions 416 to identify fiducials in the external image 1502 and in IVUS images 418a (or IVUS images 418b). For example, processor 406 can execute instructions 416 to identify vessel fiducials 702 corresponding to fiducials in IVUS images 418a and external image 1502.

As outlined above, a variety of techniques exist to identify fiducial in both internal and external imaging modalities. For example, side branch identification and matching are often used to co-register internal images to an external image. The present disclosure provides that processor 406 can execute instructions 416 to identify the fiducial and its location and identify the angle of the fiducial and store an indication of the fiducial location and angle in vessel fiducials 702. With some embodiments, processor 406 can identify the angle of the fiducial using image processing techniques and/or ML inference. For example, ML model 702 could be trained as outlined above to identify fiducials and their corresponding angle from external image 1502. Once the angle of the fiducial in the external image 1502 is identified, processor 406 can execute instructions 416 to identify an offset angle (e.g., IVUS run frame mapping 420, or the like) with which to rotate frames of the IVUS images (e.g., IVUS images 418a and/or 418b) to align the viewing angle with that of the external image 1502. Further, processor 406 can execute instructions 416 to identify an offset for other frames in the IVUS images given the offset angle of frames corresponding to the fiducials (e.g., as outlined above with respect to FIG. 10A and FIG. 10B, or the like).

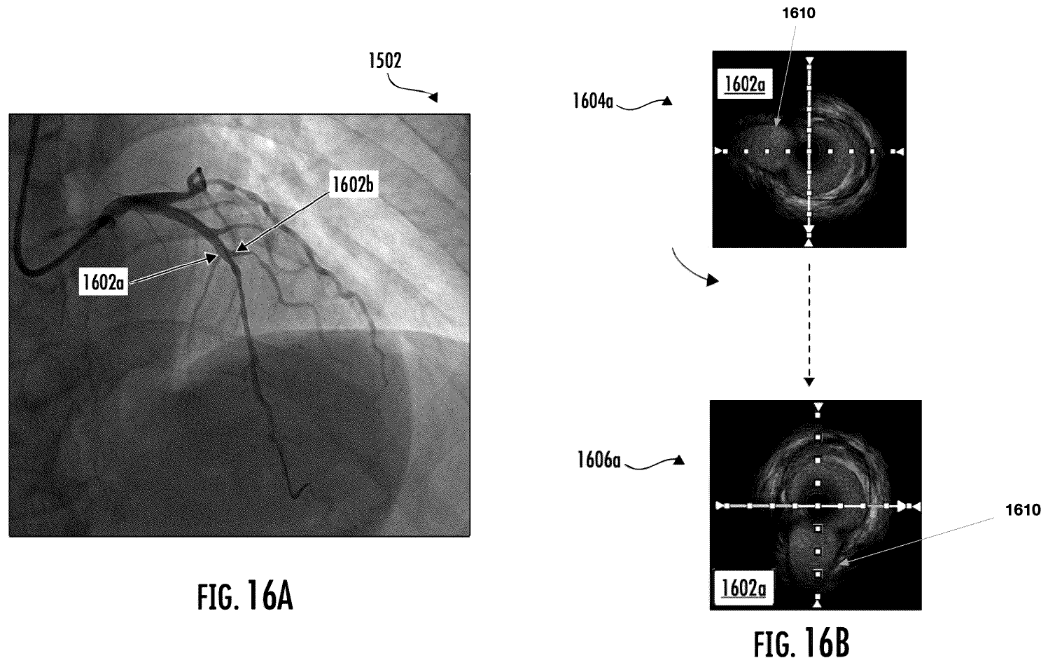

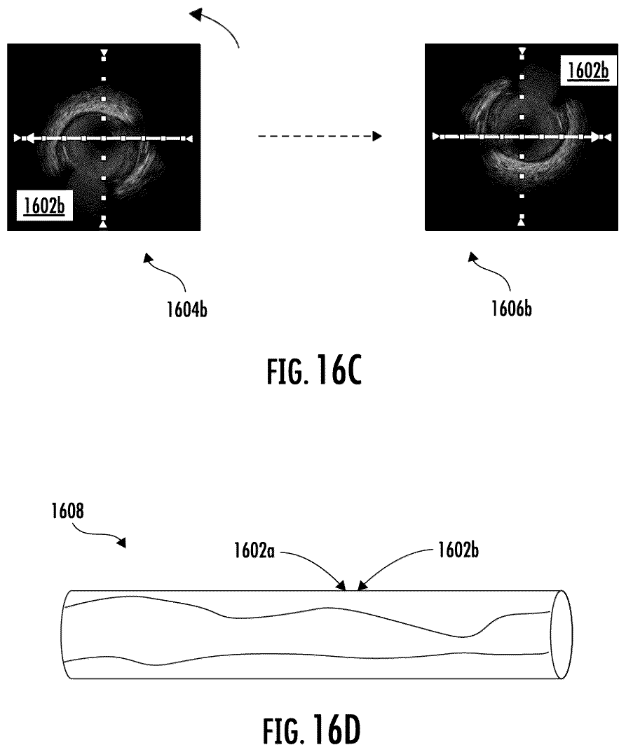

For example, FIG. 16A illustrates external image 1502 and two identified fiducials (e.g., side branches) 1602a and 1602b. Processor 406 can execute instructions 416 to identify an angle of the fiducials 1602a and 1602b. It is noted that the angle of the fiducials is derived based on a baseline, such as, setting zero (0) degrees as the Z direction from the two-dimensional (2D) image towards the viewer, or the like. Processor 406 can execute instructions 416 to rotate (or derive an angular offset) for frames from IVUS images 418a matching the fiducials 1602a and 1602b based on the angle of the fiducials 1602a and 1602b.

For example, FIG. 16B and FIG. 16C illustrate image frames 1604a and 1604b (e.g., frames from IVUS images 418a, or the like) depicting fiducials 1602a and 1602b, respectively. Processor 406 can execute instructions 416 to rotate the image frames 1604a and 1604b based on the angle of the vessel fiducials (e.g., side branches angles, or the like) represented in the external image 1502, as well as the angle of the fiducials in each respective frame 1604a and 1604b, resulting in rotated image frames 1606a and 1606b. Rotated image frames 1606a and 1606b are depicted in FIG. 16B and FIG. 16C, respectively.

In some examples, an image frame can be rotated based on a fiducial landmark. For example, a fiducial landmark 1610 is depicted in FIG. 16B. In some embodiments, processor 406 can execute instructions 416 to identify fiducial landmarks and rotate image frames based on an angle of a fiducial landmark. For example, the fiducial landmark 1610 (e.g., side branch) in image frame 1604a is depicted at approximately the 9 O'clock, or 270 degrees. This frame can be rotated an angle based on the angle of the fiducial landmark in another image frame such that the fiducial landmarks align at a particular angle. For example, rotated image frame 1606a shows the fiducial landmark rotated to 180 degrees.

Accordingly, as outlined above, processor 406 can execute instructions 416 to angularly align frames within an IVUS run with a viewing perspective of an external image (e.g., external image 1502, or the like) such that the angle in which fiducials are viewed aligns between both imaging modalities. FIG. 16D illustrates a set of external image aligned IVUS images 1608, which can correspond to frames from IVUS images 418a (or the like) where the viewing angle (or perspective) has been aligned with that of the external image frame 1502. It is noted that this provides a significant improvement over conventional techniques. It is to be appreciated that intravascular images are often agnostic to the viewing angle. For example, IVUS images are captured as the ultrasound transducer is rotated within the vessel. As such, the actual viewing angle between frames can vary. Further, the viewing angle of an external image can also vary (e.g., based on the position of the patient with respect to the image acquisition system, or the like). As such, the viewing perspective between intravascular and extravascular images will not typically align. The present disclosure addresses this issue.

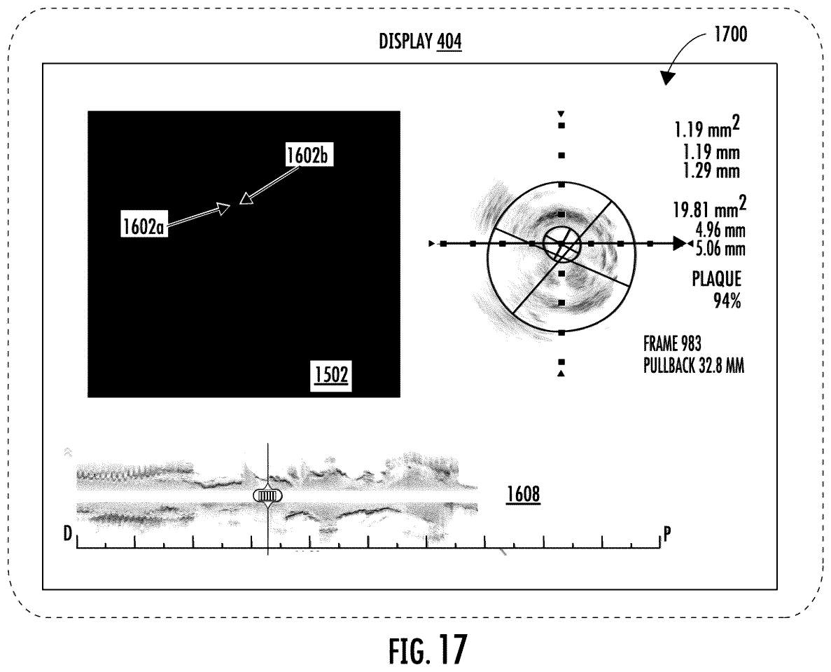

Further, as discussed above, a GUI can be generated to present graphical indications of an aligned IVUS run. For example, a GUI can be generated to present a visual representation of frames from an IVUS run aligned with a vessel as viewed in an external image. FIG. 17 illustrates a GUI 1700, which can be generated in accordance with some embodiments of the present disclosure. In some embodiments, GUI 1700 can be GUI 424 of FIG. 4, FIG. 7, or FIG. 15. For example, processor 406 can execute instructions 416 to generate GUI 424 having graphical components and an arrangement as depicted in GUI 1700 of FIG. 17. In such an example, processor 406 can execute instructions 416 to cause GUI 1700 to be displayed on display 404.

GUI 1700 can include graphical indications of external image 1502 and IVUS external image aligned IVUS images 1608. Accordingly, as a physician (or user) inspects frames of the IVUS images 418a, the external image aligned IVUS images 1608 will be presented such that the lumen and fiducials as viewed in the IVUS image frames will match the angle of the vessel and fiducials (e.g., fiducials 1602a and 1602b) as viewed in the external image frame.

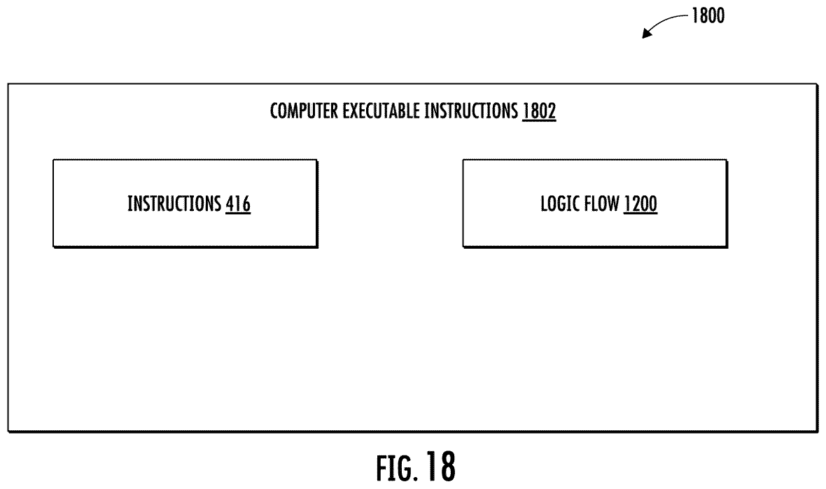

FIG. 18 illustrates computer-readable storage medium 1800. Computer-readable storage medium 1800 may comprise any non-transitory computer-readable storage medium or machine-readable storage medium, such as an optical, magnetic or semiconductor storage medium. In various embodiments, computer-readable storage medium 1800 may comprise an article of manufacture. In some embodiments, computer-readable storage medium 1800 may store computer executable instructions 1802 with which circuitry (e.g., processor 106, processor 406, processor circuit 1406, or the like) can execute. For example, computer executable instructions 1802 can include instructions to implement operations described with respect to instructions 416 and/or logic flow 1200. Examples of computer-readable storage medium 1800 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 1802 may include any suitable type of code, such as source code, compiled code, interpreted code, executable code, static code, dynamic code, object-oriented code, visual code, and the like.

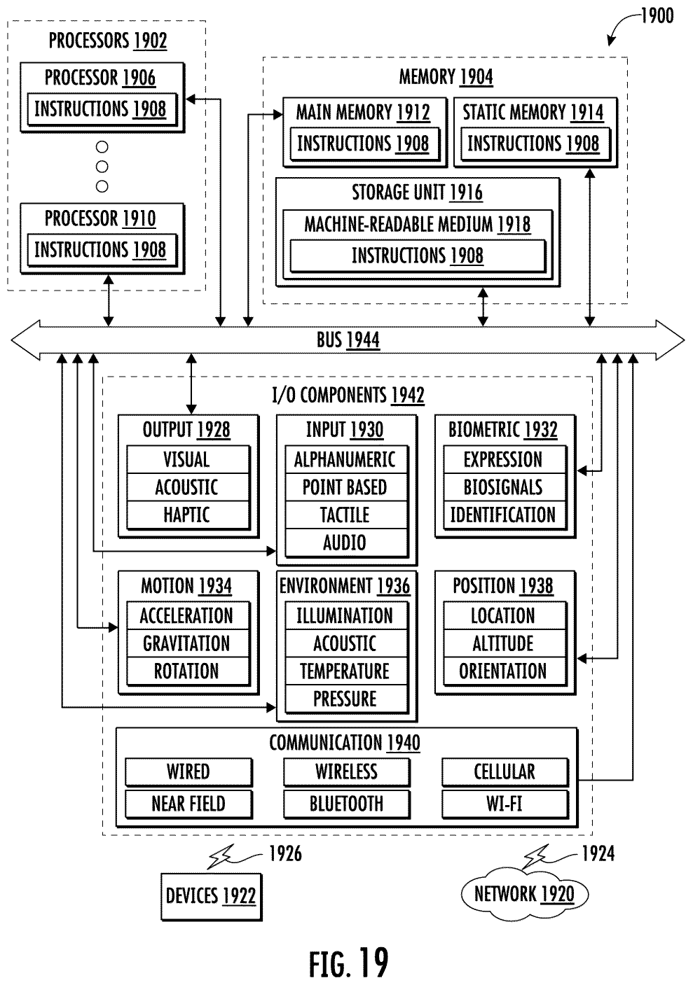

FIG. 19 illustrates a diagrammatic representation of a machine 1900 in the form of a computer system within which a set of instructions may be executed for causing the machine to perform any one or more of the methodologies discussed herein. More specifically, FIG. 19 shows a diagrammatic representation of the machine 1900 in the example form of a computer system, within which instructions 1908 (e.g., software, a program, an application, an applet, an app, or other executable code) for causing the machine 1900 to perform any one or more of the methodologies discussed herein may be executed. For example, the instructions 1908 may cause the machine 1900 to execute logic flow 1200 of FIG. 12, or the like. More generally, the instructions 1908 may cause the machine 1900 to automatically determine a mapping (e.g., in time, in distance, in angle, or the like) between frames of different IVUS runs through the same vessel (e.g., from a pre-PCI IVUS run, a peri-PCI IVUS run, and/or a post-PCI IVUS run) and/or between an IVUS run and an external image.

The instructions 1908 transform the general, non-programmed machine 1900 into a particular machine 1900 programmed to carry out the described and illustrated functions in a specific manner. In alternative embodiments, the machine 1900 operates as a standalone device or may be coupled (e.g., networked) to other machines. In a networked deployment, the machine 1900 may operate in the capacity of a server machine or a client machine in a server-client network environment, or as a peer machine in a peer-to-peer (or distributed) network environment. The machine 1900 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1908, sequentially or otherwise, that specify actions to be taken by the machine 1900. Further, while only a single machine 1900 is illustrated, the term “machine” shall also be taken to include a collection of machines 1900 that individually or jointly execute the instructions 1908 to perform any one or more of the methodologies discussed herein.

The machine 1900 may include processors 1902, memory 1904, and I/O components 1942, which may be configured to communicate with each other such as via a bus 1944. In an example embodiment, the processors 1902 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1906 and a processor 1910 that may execute the instructions 1908. The term “processor” is intended to include multi-core processors that may comprise two or more independent processors (sometimes referred to as “cores”) that may execute instructions contemporaneously. Although FIG. 19 shows multiple processors 1902, the machine 1900 may include a single processor with a single core, a single processor with multiple cores (e.g., a multi-core processor), multiple processors with a single core, multiple processors with multiples cores, or any combination thereof.

The memory 1904 may include a main memory 1912, a static memory 1914, and a storage unit 1916, both accessible to the processors 1902 such as via the bus 1944. The main memory 1904, the static memory 1914, and storage unit 1916 store the instructions 1908 embodying any one or more of the methodologies or functions described herein. The instructions 1908 may also reside, completely or partially, within the main memory 1912, within the static memory 1914, within machine-readable medium 1918 within the storage unit 1916, within at least one of the processors 1902 (e.g., within the processor's cache memory), or any suitable combination thereof, during execution thereof by the machine 1900.

The I/O components 1942 may include a wide variety of components to receive input, provide output, produce output, transmit information, exchange information, capture measurements, and so on. The specific I/O components 1942 that are included in a particular machine will depend on the type of machine. For example, portable machines such as mobile phones will likely include a touch input device or other such input mechanisms, while a headless server machine will likely not include such a touch input device. It will be appreciated that the I/O components 1942 may include many other components that are not shown in FIG. 19. The I/O components 1942 are grouped according to functionality merely for simplifying the following discussion and the grouping is in no way limiting. In various example embodiments, the I/O components 1942 may include output components 1928 and input components 1930. The output components 1928 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1930 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1942 may include biometric components 1932, motion components 1934, environmental components 1936, or position components 1938, among a wide array of other components. For example, the biometric components 1932 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 1934 may include acceleration sensor components (e.g., accelerometer), gravitation sensor components, rotation sensor components (e.g., gyroscope), and so forth. The environmental components 1936 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1938 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1942 may include communication components 1940 operable to couple the machine 1900 to a network 1920 or devices 1922 via a coupling 1924 and a coupling 1926, respectively. For example, the communication components 1940 may include a network interface component or another suitable device to interface with the network 1920. In further examples, the communication components 1940 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1922 may be another machine or any of a wide variety of peripheral devices (e.g., a peripheral device coupled via a USB).

Moreover, the communication components 1940 may detect identifiers or include components operable to detect identifiers. For example, the communication components 1940 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 1940, such as location via Internet Protocol (IP) geolocation, location via Wi-Fi® signal triangulation, location via detecting an NFC beacon signal that may indicate a particular location, and so forth.

The various memories (i.e., memory 1904, main memory 1912, static memory 1914, and/or memory of the processors 1902) and/or storage unit 1916 may store one or more sets of instructions and data structures (e.g., software) embodying or utilized by any one or more of the methodologies or functions described herein. These instructions (e.g., the instructions 1908), when executed by processors 1902, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 1920 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 1920 or a portion of the network 1920 may include a wireless or cellular network, and the coupling 1924 may be a Code Division Multiple Access (CDMA) connection, a Global System for Mobile communications (GSM) connection, or another type of cellular or wireless coupling. In this example, the coupling 1924 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 1908 may be transmitted or received over the network 1920 using a transmission medium via a network interface device (e.g., a network interface component included in the communication components 1940) and utilizing any one of several well-known transfer protocols (e.g., hypertext transfer protocol (HTTP)). Similarly, the instructions 1908 may be transmitted or received using a transmission medium via the coupling 1926 (e.g., a peer-to-peer coupling) to the devices 1922. The terms “transmission medium” and “signal medium” mean the same thing and may be used interchangeably in this disclosure. The terms “transmission medium” and “signal medium” shall be taken to include any intangible medium that can store, encoding, or carrying the instructions 1908 for execution by the machine 1900, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. An apparatus for an intravascular imaging system, comprising:
a display;
a processor coupled to the display; and
a memory device coupled to the processor, the memory device comprising instructions executable by the processor, which instructions when executed by the processor cause the intravascular imaging system to:
receive a first series of intravascular ultrasound (IVUS) images of a vessel of a patient, the first series of IVUS images comprising a first plurality of frames;
receive a second series of intravascular ultrasound (IVUS) images of the vessel of the patient, the second series of IVUS images comprising a second plurality of frames;
determine an offset for the first plurality of frames based at least in part on the second plurality of frames;
apply the offset to the first plurality of frames to generate an offset series of IVUS images;
generate a graphical user interface (GUI), the GUI comprising indications of the offset series of IVUS images and the second series of IVUS images; and
display the GUI on the display.

2. The apparatus of claim 1, wherein the instructions further cause the intravascular imaging system to:
identify a frame of the first plurality of frames comprising a vessel fiducial;
identify a frame of the second plurality of frames comprising the vessel fiducial; and
determine the offset for the first plurality of frames that when applied aligns the frame of the first plurality of frames comprising the vessel fiducial with the frame of the second plurality of frames comprising the vessel fiducial.

3. The apparatus of claim 2, wherein the offset comprises a first offset and a second offset and wherein the instructions further cause the intravascular imaging system to:
identify a first frame of the first plurality of frames comprising a first vessel fiducial;
identify a second frame of the second plurality of frames comprising the first vessel fiducial;
determine the first offset for the first plurality of frames that when applied to a first segment of the first plurality of frames aligns the first frame of the first plurality of frames with the first frame of the second plurality of frames;
identify a second frame of the first plurality of frames comprising a second vessel fiducial;
identify a second frame of the second plurality of frames comprising the second vessel fiducial; and
determine the second offset for the first plurality of frames that when applied to a second segment of the first plurality of frames different than the first segment, aligns the second frame of the first plurality of frames with the second frame of the second plurality of frames,
wherein the second offset is different from the first offset.

4. The apparatus of claim 3, wherein the first offset comprises an offset distance and the second offset comprises an offset angle or wherein the first offset comprises an offset distance or an offset angle and the second offset comprises an offset distance and an offset angle.

5. The apparatus of claim 2, wherein the instructions further cause the intravascular imaging system to:
execute a machine learning (ML) model to infer the frame of the first plurality of frames comprising the vessel fiducial; and
execute the ML model to infer the frame of the second plurality of frames comprising the vessel fiducial.

6. The apparatus of claim 5, wherein the vessel fiducial is one of a lumen geometry, a vessel geometry, a side branch location, a calcium morphology, a plaque distribution, or a guide catheter position.

7. At least one machine readable storage device, comprising a plurality of instructions that in response to being executed by a processor of an intravascular ultrasound (IVUS) imaging system cause the processor to:
receive a first series of intravascular ultrasound (IVUS) images of a vessel of a patient, the first series of IVUS images comprising a first plurality of frames;
receive a second series of intravascular ultrasound (IVUS) images of the vessel of the patient, the second series of IVUS images comprising a second plurality of frames;
determine an offset for the first plurality of frames based at least in part on the second plurality of frames;
apply the offset to the first plurality of frames to generate an offset series of IVUS images;
generate a graphical user interface (GUI), the GUI comprising indications of the offset series of IVUS images and the second series of IVUS images; and
send the GUI to a display coupled to the IVUS imaging system.

8. The at least one machine readable storage device of claim 7, wherein execution of the instructions further causes the IVUS imaging system to:
calculate a correlation score for each frame of the first plurality of frames based on a frame-by-frame correlation with the second plurality of frames;
identify a frame of the first plurality of frames having the highest correlation score and a frame of the second plurality of frames associated with the highest correlation score; and
determine the offset for the first plurality of frames that when applied aligns the frame of the first plurality of frames with the highest correlation score with the frame of the second plurality of frames associated with the highest correlation score.

9. The at least one machine readable storage device of claim 8, wherein the offset is an offset distance and wherein execution of the instructions further causes the IVUS imaging system to:
calculate a correlation score for each frame of the first plurality of frames based on an angular offset frame-by-frame correlation with the second plurality of frames;
identify a frame of the first plurality of frames having the highest correlation score and a frame or a rotated version of the frame of the second plurality of frames associated with the highest correlation score; and
determine an offset angle for the first plurality of frames based on the frame or the rotated version of the frame of the second plurality of frames associated with the highest correlation score that when applied aligns the frame of the first plurality of frames with the highest correlation score with the frame of the second plurality of frames associated with the highest correlation score,
wherein the offset series of IVUS images is generated by applying the offset distance and the offset angle to the first plurality of frames.

10. The at least one machine readable storage device of claim 7, wherein execution of the instructions further causes the IVUS imaging system to:
calculate a correlation score for each frame of the first plurality of frames based on an angular offset frame-by-frame correlation with the second plurality of frames;
identify a frame of the first plurality of frames having the highest correlation score and a frame or a rotated version of the frame of the second plurality of frames associated with the highest correlation score; and
determine the offset for the first plurality of frames based on the frame or the rotated version of the frame of the second plurality of frames associated with the highest correlation score that when applied aligns the frame of the first plurality of frames with the highest correlation score with the frame of the second plurality of frames associated with the highest correlation score.

11. The at least one machine readable storage device of claim 7, wherein the offset for the first plurality of frames is a distance offset, an angle offset, or a distance and an angle offset.

12. A method for a computing device, comprising:
receiving, by a processor, a first series of intravascular ultrasound (IVUS) images of a vessel of a patient, the first series of IVUS images comprising a first plurality of frames;
receiving, by the processor, a second series of intravascular ultrasound (IVUS) images of the vessel of the patient, the second series of IVUS images comprising a second plurality of frames;
determining, by the processor, an offset for the first plurality of frames based at least in part on the second plurality of frames;
applying, by the processor, the offset to the first plurality of frames to generate an offset series of IVUS images; and
generating, by the processor, a graphical user interface (GUI), the GUI comprising indications of the offset series of IVUS images and the second series of IVUS images.

13. The method of claim 12, wherein determining the offset for the first plurality of frames comprises:
identifying a frame of the first plurality of frames comprising a vessel fiducial;
identifying a frame of the second plurality of frames comprising the vessel fiducial; and
determining the offset for the first plurality of frames that when applied aligns the frame of the first plurality of frames comprising the vessel fiducial with the frame of the second plurality of frames comprising the vessel fiducial.

14. The method of claim 13, wherein the offset comprises a first offset and a second offset and wherein determining the offset for the first plurality of frames based comprises:
identifying a first frame of the first plurality of frames comprising a first vessel fiducial;
identifying a second frame of the second plurality of frames comprising the first vessel fiducial;
determining the first offset for the first plurality of frames that when applied to a first segment of the first plurality of frames aligns the first frame of the first plurality of frames with the first frame of the second plurality of frames;
identifying a second frame of the first plurality of frames comprising a second vessel fiducial;
identifying a second frame of the second plurality of frames comprising the second vessel fiducial; and
determining the second offset for the first plurality of frames that when applied to a second segment of the first plurality of frames different than the first segment, aligns the second frame of the first plurality of frames with the second frame of the second plurality of frames,
wherein the second offset is different from the first offset.

15. The method of claim 14, wherein the first offset comprises an offset distance and the second offset comprises an offset angle or wherein the first offset comprises an offset distance or an offset angle and the second offset comprises an offset distance and an offset angle.

16. The method of claim 13, wherein identifying the frame of the first plurality of frames comprising the vessel fiducial and wherein identifying the frame of the second plurality of frames comprising the vessel fiducial comprises:
executing a machine learning (ML) model to infer the frame of the first plurality of frames comprising the vessel fiducial; and
executing the ML model to infer the frame of the second plurality of frames comprising the vessel fiducial.

17. The method of claim 12, wherein determining the offset for the first plurality of frames comprises:
calculating a correlation score for each frame of the first plurality of frames based on a frame-by-frame correlation with the second plurality of frames;
identifying a frame of the first plurality of frames having the highest correlation score and a frame of the second plurality of frames associated with the highest correlation score; and
determining the offset for the first plurality of frames that when applied aligns the frame of the first plurality of frames with the highest correlation score with the frame of the second plurality of frames associated with the highest correlation score.

18. The method of claim 17, wherein the offset is an offset distance and wherein the method further comprises:
calculating a correlation score for each frame of the first plurality of frames based on an angular offset frame-by-frame correlation with the second plurality of frames;
identifying a frame of the first plurality of frames having the highest correlation score and a frame or a rotated version of the frame of the second plurality of frames associated with the highest correlation score; and
determining an offset angle for the first plurality of frames based on the frame or the rotated version of the frame of the second plurality of frames associated with the highest correlation score that when applied aligns the frame of the first plurality of frames with the highest correlation score with the frame of the second plurality of frames associated with the highest correlation score,
wherein the offset series of IVUS images is generated by applying the offset distance and the offset angle to the first plurality of frames.

19. The method of claim 12, wherein determining the offset for the first plurality of frames comprises:
calculating a correlation score for each frame of the first plurality of frames based on an angular offset frame-by-frame correlation with the second plurality of frames;
identifying a frame of the first plurality of frames having the highest correlation score and a frame or a rotated version of the frame of the second plurality of frames associated with the highest correlation score; and
determining the offset for the first plurality of frames based on the frame or the rotated version of the frame of the second plurality of frames associated with the highest correlation score that when applied aligns the frame of the first plurality of frames with the highest correlation score with the frame of the second plurality of frames associated with the highest correlation score.

20. The method of claim 12, wherein the offset for the first plurality of frames is a distance offset, an angle offset, or a distance and an angle offset.
